SEQUENCE LISTING (39 sequences)

SEQ ID NO: 1 - protein, 505 aa, synthetic construct
  REGION 1..505: note = PD-L1 TriKE amino acid sequence
QVQLVESGGGLVQPGGSLRLSCAASGLTFSSYNMGWFRQAPGQGLEAVASITWSGRDTFYADSVKGRFTISRDNSKNTLYLQMNSLRAEDTAVYYCAANPWPVAAPRSGTYWGQGTLVTVSSSGGGGSGGGGSGGGGSGGGGSGNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSTSGSGKPGSGEGSTKGEIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKQVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSS

SEQ ID NO: 2 - protein, 122 aa, synthetic construct
  REGION 1..122: note = CAM CD16
QVQLVESGGGLVQPGGSLRLSCAASGLTFSSYNMGWFRQAPGQGLEAVASITWSGRDTFYADSVKGRFTISRDNSKNTLYLQMNSLRAEDTAVYYCAANPWPVAAPRSGTYWGQGTLVTVSS

SEQ ID NO: 3 - protein, 22 aa, synthetic construct
  REGION 1..22: note = Linkerseq 16 Linker
SGGGGSGGGGSGGGGSGGGGSG

SEQ ID NO: 4 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 5 - protein, 18 aa, synthetic construct
  REGION 1..18: note = IL-15 (wild-type)
GSTSGSGKPGSGEGSTKG

SEQ ID NO: 6 - protein, 106 aa, synthetic construct
  REGION 1..106: note = anti-PD-L1 light chain (human)
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIK

SEQ ID NO: 7 - protein, 123 aa, synthetic construct
  REGION 1..123: note = anti-PD-L1 heavy chain (human)
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSS

SEQ ID NO: 8 - DNA, 1515 bp, synthetic construct
  misc_feature 1..1515: note = DNA encoding PD-L1 TriKE
  source 1..1515: mol_type = other DNA
caggtgcagctggtggagtctgggggaggcttggtgcagcctgggggctctctgagactctcctgtgcagcctctggcctcaccttcagtagctataacatgggctggttccgccaggctccagggcaaggccttgaggctgtagcatctattacctggagtggtcgggacacattctatgcagactccgtgaagggccgattcaccatctccagagacaactccaagaacactctctatctgcaaatgaacagcctgcgcgcggaggacacggccgtttattattgtgctgcaaacccctggccagtggcggcgccacgtagtggcacctactggggccaagggaccctggtcaccgtctcctcatctggcggcggcggttctggtggaggaggtagtggggggggaggaagcggagggggtggctcagggaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctggcagtaccagcgggtcagggaaacctggcagtggggaaggttccacaaaaggtgaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggccgacgttcggccaagggaccaaggtggaaatcaaacaggtccaactggtgcagtctggggctgaggtcaagaagcctgggtcgtcggtgaaggtctcctgcaagacttctggagacaccttcagcacctatgctatcagctgggtgcgacaggcccctggacaagggcttgagtggatgggagggatcatccctatatttggtaaagcacactacgcacagaagttccagggcagagtcacgattaccgcggacgaatccacgagcacagcctacatggagctgagcagcctgagatctgaggacacggccgtgtatttttgtgcgagaaagtttcactttgtttcggggagccccttcggtatggacgtctggggccaagggaccacggtcaccgtctcctca

SEQ ID NO: 9 - DNA, 366 bp, synthetic construct
  misc_feature 1..366: note = DNA encoding CAM CD16
  source 1..366: mol_type = other DNA
caggtgcagctggtggagtctgggggaggcttggtgcagcctgggggctctctgagactctcctgtgcagcctctggcctcaccttcagtagctataacatgggctggttccgccaggctccagggcaaggccttgaggctgtagcatctattacctggagtggtcgggacacattctatgcagactccgtgaagggccgattcaccatctccagagacaactccaagaacactctctatctgcaaatgaacagcctgcgcgcggaggacacggccgtttattattgtgctgcaaacccctggccagtggcggcgccacgtagtggcacctactggggccaagggaccctggtcaccgtctcctca

SEQ ID NO: 10 - DNA, 66 bp, synthetic construct
  misc_feature 1..66: note = DNA encoding Linker16 linker
  source 1..66: mol_type = other DNA
tctggcggcggcggttctggtggaggaggtagtggggggggaggaagcggagggggtggctcaggg

SEQ ID NO: 11 - DNA, 342 bp, synthetic construct
  misc_feature 1..342: note = DNA encoding IL-15 (wt)
  source 1..342: mol_type = other DNA
aactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttct

SEQ ID NO: 12 - DNA, 54 bp, synthetic construct
  misc_feature 1..54: note = DNA encoding Whitlow Linker
  source 1..54: mol_type = other DNA
ggcagtaccagcgggtcagggaaacctggcagtggggaaggttccacaaaaggt

SEQ ID NO: 13 - DNA, 318 bp, synthetic construct
  misc_feature 1..318: note = DNA encoding anti-PD-L1 Light chain
  source 1..318: mol_type = other DNA
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggccgacgttcggccaagggaccaaggtggaaatcaaa

SEQ ID NO: 14 - DNA, 369 bp, synthetic construct
  misc_feature 1..369: note = DNA encoding anti-PD-L1 Heavy chain
  source 1..369: mol_type = other DNA
caggtccaactggtgcagtctggggctgaggtcaagaagcctgggtcgtcggtgaaggtctcctgcaagacttctggagacaccttcagcacctatgctatcagctgggtgcgacaggcccctggacaagggcttgagtggatgggagggatcatccctatatttggtaaagcacactacgcacagaagttccagggcagagtcacgattaccgcggacgaatccacgagcacagcctacatggagctgagcagcctgagatctgaggacacggccgtgtatttttgtgcgagaaagtttcactttgtttcggggagccccttcggtatggacgtctggggccaagggaccacggtcaccgtctcctca

SEQ ID NO: 15 - protein, 492 aa, synthetic construct
  REGION 1..492: note = PD-L1 TriKE amino acid sequence
QVQLVESGGGLVQPGGSLRLSCAASGLTFSSYNMGWFRQAPGQGLEAVASITWSGRDTFYADSVKGRFTISRDNSKNTLYLQMNSLRAEDTAVYYCAANPWPVAAPRSGTYWGQGTLVTVSSPSGQAGAAASESLFVSNHAYNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSEASGGPEEIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKQVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSS

SEQ ID NO: 16 - protein, 20 aa, synthetic construct
  REGION 1..20: note = HMA Linker
PSGQAGAAASESLFVSNHAY

SEQ ID NO: 17 - protein, 7 aa, synthetic construct
  REGION 1..7: note = Linker
EASGGPE

SEQ ID NO: 18 - DNA, 1476 bp, synthetic construct
  misc_feature 1..1476: note = DNA encoding PD-L1 TriKE
  source 1..1476: mol_type = other DNA
caggtgcagctggtggagtctgggggaggcttggtgcagcctgggggctctctgagactctcctgtgcagcctctggcctcaccttcagtagctataacatgggctggttccgccaggctccagggcaaggccttgaggctgtagcatctattacctggagtggtcgggacacattctatgcagactccgtgaagggccgattcaccatctccagagacaactccaagaacactctctatctgcaaatgaacagcctgcgcgcggaggacacggccgtttattattgtgctgcaaacccctggccagtggcggcgccacgtagtggcacctactggggccaagggaccctggtcaccgtctcctcaccgtctggtcaggctggtgctgctgctagcgaatctctgttcgtttctaaccacgcttacaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctgaagcttccggaggtcccgaggaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggccgacgttcggccaagggaccaaggtggaaatcaaacaggtccaactggtgcagtctggggctgaggtcaagaagcctgggtcgtcggtgaaggtctcctgcaagacttctggagacaccttcagcacctatgctatcagctgggtgcgacaggcccctggacaagggcttgagtggatgggagggatcatccctatatttggtaaagcacactacgcacagaagttccagggcagagtcacgattaccgcggacgaatccacgagcacagcctacatggagctgagcagcctgagatctgaggacacggccgtgtatttttgtgcgagaaagtttcactttgtttcggggagccccttcggtatggacgtctggggccaagggaccacggtcaccgtctcctca

SEQ ID NO: 19 - DNA, 60 bp, synthetic construct
  misc_feature 1..60: note = DNA encoding HMA linker
  source 1..60: mol_type = other DNA
ccgtctggtcaggctggtgctgctgctagcgaatctctgttcgtttctaaccacgcttac

SEQ ID NO: 20 - DNA, 21 bp, synthetic construct
  misc_feature 1..21: note = DNA encoding Linker
  source 1..21: mol_type = other DNA
gaagcttccggaggtcccgag

SEQ ID NO: 21 - protein, 114 aa, synthetic construct
  REGION 1..114: note = Altor IL-15 N72D mutation amino acid sequence
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 22 - protein, 114 aa, synthetic construct
  REGION 1..114: note = Altor IL-15 N72A mutation amino acid sequence
NWVNVISDLKKIEDLIQSMHIDATLYTESDVHPSCKVTAMKCFLLELQVISLESGDASIHDTVENLIILANASLSSNGNVTESGCKECEELEEKNIKEFLQSFVHIVQMFINTS

SEQ ID NO: 23 - protein, 240 aa, Homo sapiens
MEVQLVESGGGVVRPGGSLRLSCAASGFTFDDYGMSWVRQAPGKGLEWVSGINWNGGSTGYADSVKGRFTISRDNAKNSLYLQMNSLRAEDTAVYYCARGRSLLFDYWGQGTLVTVSRGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVL

SEQ ID NO: 24 - DNA, 720 bp, Homo sapiens
  source 1..720: mol_type = other DNA
atggaagtgcagctggtggaaagcggcggcggcgtggtgcgcccgggcggcagcctgcgcctgagctgcgcggcgagcggctttacctttgatgattatggcatgagctgggtgcgccaggcgccgggcaaaggcctggaatgggtgagcggcattaactggaacggcggcagcaccggctatgcggatagcgtgaaaggccgctttaccattagccgcgataacgcgaaaaacagcctgtatctgcagatgaacagcctgcgcgcggaagataccgcggtgtattattgcgcgcgcggccgcagcctgctgtttgattattggggccagggcaccctggtgaccgtgagccgcggcggcggcggcagcggcggcggcggcagcggcggcggcggcagcagcgaactgacccaggatccggcggtgagcgtggcgctgggccagaccgtgcgcattacctgccagggcgatagcctgcgcagctattatgcgagctggtatcagcagaaaccgggccaggcgccggtgctggtgatttatggcaaaaacaaccgcccgagcggcattccggatcgctttagcggcagcagcagcggcaacaccgcgagcctgaccattaccggcgcgcaggcggaagatgaagcggattattattgcaacagccgcgatagcagcggcaaccatgtggtgtttggcggcggcaccaaactgaccgtgctg

SEQ ID NO: 25 - protein, 475 aa, synthetic construct
  REGION 1..475: note = HLE sequence 1
DKTHTCPPCPAPELLGGPSVFLFPPKPKDTLMISRTPEVTCVWDVSHEDPEVKFNWYVDGVEVHNAKTKPCEEQYNSYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCWVDVSHEDPEVKFNWYDGVEVHNAKTKPCEEQYNSTYRCVSVLTVLHQDWLNGKEYKCKVSNKALPAPIEKTISKAKGQPREPQVYTLPPSREEMKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 26 - protein, 482 aa, synthetic construct
  REGION 1..482: note = HLE sequence 2
DKTHTCPPCPAPELLGGPSVFLFPPKPKDTLMISRTPEVTCVWDVSHEDPEVKFNWYVDGVEVHNAKTKPCEEQYNSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCWVDVSHEDPEVKFNWYVDGVEVHNAKTKPCEEQYNSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 226 aa, synthetic construct
  REGION 1..226: note = HLE sequence 3
DKTHTCPPCPAPELLGGPSVFLFPPKPKDTLMISRTPEVTCWVDVSHEDPEVKFNWYVDGVEVHNAKTKPCEEQYGSTYRCVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 28 - protein, 226 aa, synthetic construct
  REGION 1..226: note = LE sequence 4
DKTHTCPPCPAPELLGGPSVFLFPPKPKDTLMISRTPEVTCVWDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 29 - protein, 482 aa, synthetic construct
  REGION 1..482: note = HLE sequence 5
DKTHTCPPCPAPELLGGPSVFLFPPKPKDTLMISRTPEVTCWVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVWDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 223 aa, synthetic construct
  REGION 1..223: note = Fc region 1
DKTHTCPPCPAPELLGGPSVFLFPPKPKDTLMISRTPEVTCVWDVSHEDPEVKFNWYVDGVEVHNAKTKPCEEQYNSYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 31 - protein, 222 aa, synthetic construct
  REGION 1..222: note = Fc region 2
DKTHTCPPCPAPELLGGPSVFLFPPKPKDTLMISRTPEVTCWVDVSHEDPEVKFNWYDGVEVHNAKTKPCEEQYNSTYRCVSVLTVLHQDWLNGKEYKCKVSNKALPAPIEKTISKAKGQPREPQVYTLPPSREEMKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 32 - protein, 226 aa, synthetic construct
  REGION 1..226: note = Fc region 3
DKTHTCPPCPAPELLGGPSVFLFPPKPKDTLMISRTPEVTCVWDVSHEDPEVKFNWYVDGVEVHNAKTKPCEEQYNSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 226 aa, synthetic construct
  REGION 1..226: note = Fc region 4
DKTHTCPPCPAPELLGGPSVFLFPPKPKDTLMISRTPEVTCWVDVSHEDPEVKFNWYVDGVEVHNAKTKPCEEQYNSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 226 aa, synthetic construct; identical to SEQ ID NO: 27
  REGION 1..226: note = Fc region 5

SEQ ID NO: 35 - protein, 226 aa, synthetic construct; identical to SEQ ID NO: 28
  REGION 1..226: note = Fc region 6

SEQ ID NO: 36 - protein, 226 aa, synthetic construct
  REGION 1..226: note = Fc region 7
DKTHTCPPCPAPELLGGPSVFLFPPKPKDTLMISRTPEVTCWVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 226 aa, synthetic construct
  REGION 1..226: note = Fc region 8
DKTHTCPPCPAPELLGGPSVFLFPPKPKDTLMISRTPEVTCVWDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 30 aa, synthetic construct
  REGION 1..30: note = scFc linker 1
GGGGSGGGGSGGGGSGGGGSGGGGSGGGGS

SEQ ID NO: 39 - protein, 17 aa, synthetic construct
  REGION 1..17: note = scFc linker 2
SSGGGGSGGGGSGGGGS

(19) United States
(12) Patent Application Publication
(10) Pub. No.: US 2024/0425597 A1
(43) Pub. Date: Dec. 26, 2024

(54) PD-L1 TARGETING FUSION PROTEINS AND METHODS OF USE THEREOF

(71) Applicant: GT Biopharma, Inc., Brisbane, CA (US)

(72) Inventor: Martin Schroeder, Brisbane, CA (US)

(21) Appl. No.: 18/692,775

(22) PCT Filed: Sep. 15, 2022
(86) PCT No.: PCT/US2022/043711

§ 371 Date: Mar. 15, 2024

Related U.S. Application Data
(60) Provisional application No. 63/245,135, filed on Sep. 16, 2021.

(51) Int. Cl. C07K 16/28

(52) U.S. Cl. CPC C07K 16/2827; C07K 2319/00

(57) ABSTRACT

The invention provides PD-L1 targeting fusion proteins and methods of use thereof. The targeting fusion proteins include tri-specific killer engager molecules including a camelid or a human CD16 natural killer (NK) cell engager domain, a wild type or a mutant IL-15 cytokine NK activating domain and a light chain and a heavy chain of a PD-L1 targeting peptide. The methods of use include methods of treating cancer.

### CROSS-REFERENCE TO RELATED APPLICATIONS

This application claims benefit of priority under 35 U.S.C. § 119 (c) of U.S. Provisional Application No. 63/245,135, filed Sep. 16, 2021. The disclosure of the prior application is considered part of and are herein incorporated by reference in the disclosure of this application in its entirety.

### INCORPORATION OF SEQUENCE LISTING

The material in the accompanying sequence listing is hereby incorporated by reference into this application. The accompanying sequence listing xml file, name G1421US00_GTBIO2190-1WO.xml, was created on Sep. 9, 2022 and is 58kb in size.

### BACKGROUND OF THE INVENTION

### Field of the Invention

The present invention relates generally to fusion proteins, and more specifically to PD-L1 targeting tri-specific killer engager molecules and their use to treat cancer.

### Background Information

Immunotherapy is an individualized treatment that activates or suppresses the immune system to amplify or diminish an immune response and is developing rapidly for treating various forms of cancer. Immunotherapy for cancer, such as chimeric antigen receptor (CAR)-T cells, CAR-natural killer (NK) cells, PD-1 and PD-L1 inhibitor, aims to help patients' immune system fight cancer. The activation of T cell depends on both the specific combination of T cell receptor (TCR) and peptide-bound major histocompatibility complex (MHC), and the interplay of co-stimulatory molecules of T cell with ligands on antigen presenting cells (APCs).

Immune checkpoints, such as PD-1, PD-L1, PD-L2, and CTLA4, are molecules holding many receptor-ligand interactions to evade the immune system and facilitate proliferation. Several monoclonal antibodies (mAbs) that block these proteins were developed to down-regulate the inhibitory immune response and promote the cellular cytotoxicity of T cells that eliminate tumor cells. Among the immune checkpoint-blocking drugs, the inhibitors targeting PD-1 or CTLA4 were successfully used for treating patients with metastatic melanoma, with improved responses and prolonged survival. This success led to the development of such agents for treating a wide range of malignancies, including renal cell carcinoma (RCC), NSCLC, and acute myeloid leukemia (AML), which further enhanced the response rate compared to conventional treatments, and prolonged the survival time of patients (Yang et al., Int J Biol Sci 2020; 16 (11): 1767-1773).

Programmed cell death-1 receptor (PD-1) and its ligands (PD-L1/PD-L2) belong to the family of immune checkpoint proteins that act as co-inhibitory factors that can halt or limit the development of the T cell response. PD-1 is expressed on the surface of activated T cells, while PD-L1 and PD-L2 are expressed on the surface of dendritic cells or macrophages. PD-1/PD-L1 interaction ensures that the immune system is activated only at the appropriate time in order to minimize the possibility of chronic autoimmune inflammation. Under normal conditions, the immune system performs a series of steps which lead to an anticancer immune response and cancer cell death based on the activation of T cells by dendritic cells presenting tumor-antigens, and the release by T cells of cytotoxins which induce apoptosis in their target cancer cells.

The PD-1/PD-L1 pathway represents an adaptive immune resistance mechanism exerted by tumor cells in response to endogenous immune anti-tumor activity. PD-L1 is overexpressed on tumor cells or on non-transformed cells in the tumor microenvironment. PD-L1 expressed on the tumor cells binds to PD-1 receptors on the activated T cells, which leads to the inhibition of the cytotoxic T cells. These deactivated T cells remain inhibited in the tumor microenvironment.

Despite certain successes, there are limitations that decrease the overall efficiency of mAb therapies. With the development of CD16-directed bispecific and tri-specific single-chain fragment variable (BiKEs and TriKEs) recombinant molecules, most of these undesired limitations are avoided while eliciting high effector function as they lack the Fc portion of whole antibodies and have a targeted specificity for CD16 (Gleason et al., Mol Cancer Ther; 11 (12); 2674-84, 2012). As a result, recombinant reagents are attractive for clinical use in enhancing natural killer (NK) cell immunotherapies.

The ability of NK cells to recognize and kill targets is regulated by a sophisticated repertoire of inhibitory and activating cell surface receptors. NK cell cytotoxicity can occur by natural cytotoxicity, mediated via the natural cytotoxicity receptors (NCR), or by antibodies, such as rituximab, to trigger antibody-dependent cell-mediated cytotoxicity (ADCC) through CD16, the activating low-affinity Fc-y receptor for immunoglobulin G (IgG) highly expressed by the CD56dim subset of NK cells. CD16/CD19 BIKE and CD16/CD19/CD22 TriKE can trigger NK cell activation through direct signaling of CD16 and induce directed secretion of lytic granules and target cell death. Furthermore, these reagents induce NK cell activation that leads to cytokine and chemokine production.

### SUMMARY OF THE INVENTION

The present invention is based on the development of PD-L1 targeting fusion proteins, and specifically PD-L1 targeting tri-specific killer engager molecules (TrikEs).

In one embodiment, the present invention provides an isolated nucleic acid sequence as set forth in SEQ ID NO:13 or 14 or a sequence having 90% identity thereto.

In another embodiment, the invention provides a protein encoded by a nucleic acid sequence as set forth in SEQ ID NO: 13 or 14 or a sequence having 90% identity thereto. In one aspect, the amino acid sequence is selected from SEQ ID NO:6 or 7.

In an additional embodiment, the invention provides a fusion protein including the amino acid sequence set forth in SEQ ID NO:6 and 7, operably linked to each other in either orientation.

In one aspect, the protein includes SEQ ID NO:6 and 7, in direct linkage between the C-terminus of SEQ ID NO:6 and the N-terminus of SEQ ID NO:7. In another aspect, the protein includes SEQ ID NO:7 and 6, in direct linkage between the C-terminus of SEQ ID NO: 7 and the N-terminus of SEQ ID NO:6.

In a further embodiment, the invention provides a fusion protein including the sequence set forth in SEQ ID NO:1 or 15 and sequences having 90% or greater identity to SEQ ID NO: 1 or 15.

In one embodiment, the present invention provides a fusion protein including in operably linkage, SEQ ID NO:2 or 23; 4, 21 or 22; 6 and 7 or 7 and 6.

In one aspect, SEQ ID NO:2 or 23 and 4, 21 or 22 are linked by SEQ ID NO:3 or SEQ ID NO:16. In another aspect, SEQ ID NO:4, 17 or 18 and 6 or 7 are linked by SEQ ID NO: 5 or SEQ ID NO:17. In other aspects, SEQ ID NO:6 and 7 are in operable linkage in either orientation. In some aspects, the fusion protein further includes a half-life extending (HLE) molecule. In one aspect, the HLE molecule is a Fc or a scFc antibody fragment including any one of SEQ ID NOs: 25-29. In some aspects, SEQ ID NO:4 has an N72 substitution. In various aspects, the N72 mutation is N72A or N72D. In one aspect, the protein is set forth in SEQ ID NO: 21 or 22.

In one embodiment, the invention provides a fusion protein including SEQ ID NO: 23, SEQ ID NO:21 or 22 and SEQ ID NO:6 and 7 in either orientation. In one aspect, SEQ ID NO: 23 is operably linked to SEQ ID NO:21 or 22 by a linker of SEQ ID NO:3 or 16. In another aspect, SEQ ID NO:21 or 22 is operably linked to SEQ 6 and 7, in either orientation by a linker of SEQ ID NO:5 or 17. In some aspects, the fusion protein further includes a half-life extending (HLE) molecule. In one aspect, the HLE molecule is a Fc or a scFc antibody fragment including any one of SEQ ID NOs: 25-29.

In an additional embodiment, the invention provides an isolated nucleic acid sequence encoding any of the fusion proteins described herein.

In one aspect, the sequence is SEQ ID NO:8 or 18.

In another embodiment, the invention provides a method of treating cancer in a subject including administering to the subject any of the fusion proteins described herein, thereby treating the cancer.

In one aspect, the cancer is selected from non-small lung cancer, cutaneous squamous cell carcinoma, pancreatic cancer, primary hepatocellular carcinoma, colorectal carcinoma, clear cell renal carcinoma, prostate cancer, cervical cancer, ovarian cancer, melanoma, brain cancer, leukemia, lymphoma, myeloma, head and neck cancer or breast cancer. In some aspects, an immune checkpoint inhibitor is further administered to the subject. In various aspects, the immune checkpoint inhibitor is selected from the group consisting of programmed cell death 1 protein (PD-1) inhibitor, PD-1 ligand 1 (PD-L1) inhibitor, PDD-L2 inhibitor, cytotoxic T-lymphocyte-associated protein 4 (CTLA-4) inhibitor, adenosine A2A receptor (A2AR) inhibitor, B7-H3 inhibitor, B7-H4 inhibitor, B and T lymphocyte attenuator (BTLA) inhibitor, indoleamine 2,3-dioxygenase (IDO) inhibitor, killer-cell immunoglobulin-like receptor (KIR) inhibitor, lymphocyte activation gene-3 (LAG3) inhibitor, nicotinamide adenine dinucleotide phosphate NADPH oxidase isoform 2 (NOX2) inhibitor, sialic acid-binding immunoglobulin-type lectin 7 (SIGLEC7) inhibitor, SIGLEC9 inhibitor, T-cell immunoglobulin domain and mucin domain 3 (TIM-3) inhibitor, and V-domain Ig suppressor of T cell activation (VISTA) inhibitor.

### DETAILED DESCRIPTION OF THE INVENTION

The present invention is based on the development of PD-L1 targeting fusion proteins, and specifically PD-L1 targeting tri-specific killer engager molecules (TrikEs).

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

In one embodiment, the present invention provides an isolated nucleic acid sequence as set forth in SEQ ID NO: 13 or 14 or a sequence having 90% identity thereto.

As used herein, the term “nucleic acid” or “oligonucleotide” refers to polynucleotides such as deoxyribonucleic acid (DNA) or ribonucleic acid (RNA). Nucleic acids include but are not limited to genomic DNA, cDNA, mRNA, iRNA, miRNA, tRNA, ncRNA, rRNA, and recombinantly produced and chemically synthesized molecules such as aptamers, plasmids, anti-sense DNA strands, shRNA, ribozymes, nucleic acids conjugated and oligonucleotides. According to the invention, a nucleic acid may be present as a single-stranded or double-stranded and linear or covalently circularly closed molecule. A nucleic acid can be isolated. The term “isolated nucleic acid” means, that the nucleic acid (i) was amplified in vitro, for example via polymerase chain reaction (PCR), (ii) was produced recombinantly by cloning, (iii) was purified, for example, by cleavage and separation by gel electrophoresis, (iv) was synthesized, for example, by chemical synthesis, or (vi) extracted from a sample. A nucleic might be employed for introduction into, i.e. transfection of, cells, in particular, in the form of RNA which can be prepared by in vitro transcription from a DNA template. The RNA can moreover be modified before application by stabilizing sequences, capping, and polyadenylation.

As used herein “amplified DNA” or “PCR product” refers to an amplified fragment of DNA of defined size. Various techniques are available and well known in the art to detect PCR products. PCR product detection methods include, but are not restricted to, gel electrophoresis using agarose or polyacrylamide gel and adding ethidium bromide staining (a DNA intercalant), labeled probes (radioactive or non-radioactive labels, southern blotting), labeled deoxyribonucleotides (for the direct incorporation of radioactive or non-radioactive labels) or silver staining for the direct visualization of the amplified PCR products; restriction endonuclease digestion, that relies agarose or polyacrylamide gel or High-performance liquid chromatography (HPLC); dot blots, using the hybridization of the amplified DNA on specific labeled probes (radioactive or non-radioactive labels); high-pressure liquid chromatography using ultraviolet detection; electro-chemiluminescence coupled with voltage-initiated chemical reaction/photon detection; and direct sequencing using radioactive or fluorescently labeled deoxyribonucleotides for the determination of the precise order of nucleotides with a DNA fragment of interest, oligo ligation assay (OLA), PCR, qPCR, DNA sequencing, fluorescence, gel electrophoresis, magnetic beads, allele specific primer extension (ASPE) and/or direct hybridization.

Generally, nucleic acid can be extracted, isolated, amplified, or analyzed by a variety of techniques such as those described by Green and Sambrook, Molecular Cloning: A Laboratory Manual (Fourth Edition), Cold Spring Harbor Laboratory Press, Woodbury, NY 2,028 pages (2012); or as described in U.S. Pat. Nos. 7,957,913; 7,776,616; 5,234,809; U.S. Pub. 2010/0285578; and U.S. Pub. 2002/0190663. Examples of nucleic acid analysis include, but are not limited to, sequencing and DNA-protein interaction. Sequencing may be by any method known in the art. DNA sequencing techniques include classic dideoxy sequencing reactions (Sanger method) using labeled terminators or primers and gel separation in slab or capillary, and next generation sequencing methods such as sequencing by synthesis using reversibly terminated labeled nucleotides, pyrosequencing, 454 sequencing, Illumina/Solexa sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLID sequencing. Separated molecules may be sequenced by sequential or single extension reactions using polymerases or ligases as well as by single or sequential differential hybridizations with libraries of probes.

The terms “sequence identity” or “percent identity” are used interchangeably herein. To determine the percent identity of two polypeptide molecules or two polynucleotide sequences, the sequences are aligned for optimal comparison purposes (e.g., gaps can be introduced in the sequence of a first polypeptide or polynucleotide for optimal alignment with a second polypeptide or polynucleotide sequence). The amino acids or nucleotides at corresponding amino acid or nucleotide positions are then compared. When a position in the first sequence is occupied by the same amino acid or nucleotide as the corresponding position in the second sequence, then the molecules are identical at that position. The percent identity between the two sequences is a function of the number of identical positions shared by the sequences (i.e., % identity=number of identical positions/total number of positions (i.e., overlapping positions)×100). In some embodiments the length of a reference sequence (e.g. SEQ ID NO:13 or 14) aligned for comparison purposes is at least 80% of the length of the comparison sequence, and in some embodiments is at least 90% or 100%. In an embodiment, the two sequences are the same length.

Ranges of desired degrees of sequence identity are approximately 80% to 100% and integer values in between. Percent identities between a disclosed sequence and a claimed sequence can be at least 80%, at least 85%, at least 90%, at least 95%, at least 96%, at least 97%, at least 98%, at least 99%, at least 99.5%, or at least 99.9%. In general, an exact match indicates 100% identity over the length of the reference sequence (e.g., SEQ ID NO: 13 or 14). Preferably, sequences that are not 100% identical to sequences provided herein retain the function of the original sequence (e.g., ability to bind PD-L1 or CD16).

Polypeptides and polynucleotides that are about 85, 86, 87, 88, 89, 90, 91, 92, 93, 94, 95, 96, 97, 98, 99 99.5% or more identical to polypeptides and polynucleotides described herein are embodied within the disclosure. For example, a polypeptide can have 80%, 85%, 90%, 91%, 92%, 93%, 94%, 95%, 96%, 97%, 98%, 99% or more identity to SEQ ID NO:13 or 14.

Variants of the disclosed sequences also include peptides, or full-length protein, that contain substitutions, deletions, or insertions into the protein backbone, that would still leave at least about 70% homology to the original protein over the corresponding portion. A yet greater degree of departure from homology is allowed if like-amino acids, i.e. conservative amino acid substitutions, do not count as a change in the sequence. Examples of conservative substitutions involve amino acids that have the same or similar properties. Illustrative amino acid conservative substitutions include the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine, glutamine, or glutamate; methionine to leucine or isoleucine; phenylalanine to tyrosine, leucine or methionine; serine to threonine; threonine to serine; tryptophan to tyrosine; tyrosine to tryptophan or phenylalanine; valine to isoleucine to leucine.

In another embodiment, the invention provides a protein encoded by a nucleic acid sequence as set forth in SEQ ID NO: 13 or 14 or a sequence having 90% identity thereto.

The terms “peptide”, “polypeptide” and “protein” are used interchangeably herein and refer to any chain of at least two amino acids, linked by a covalent chemical bound. As used herein polypeptide can refer to the complete amino acid sequence coding for an entire protein or to a portion thereof. A “protein coding sequence” or a sequence that “encodes” a particular polypeptide or peptide, is a nucleic acid sequence that is transcribed (in the case of DNA) and is translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the coding sequence.

In one aspect, the amino acid sequence is selected from SEQ ID NO:6 or 7.

The nucleic acid sequences provided herein can encode for example a light chain or a heavy chain of an antibody, conferring to the encoded polypeptide a binding domain or targeting domain to a specific target. Such a polypeptide can be referred to as a targeting peptide.

The term “antibody” generally refers to immunoglobulin molecules and immunologically active portions of immunoglobulin molecules, i.e., molecules that contain an antigen binding site that immunospecifically binds an antigen. “Native antibodies” and “intact immunoglobulins”, or the like, are usually heterotetrameric glycoproteins of about 150,000 daltons, composed of two identical light (L) chains and two identical heavy (H) chains. The light chains from any vertebrate species can be assigned to one of two clearly distinct types, called kappa (κ) and lambda (λ), based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2. The heavy-chain constant domains that correspond to the different classes of immunoglobulins are called α, δ, ϵ, γ, and μ, respectively. The subunit structures and three-dimensional configurations of different classes of immunoglobulins are well known.

In a typical antibody molecule, each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light-chain variable domain is aligned with the variable domain of the heavy chain. Particular amino acid residues are believed to form an interface between the light-and heavy-chain variable domains. Each variable region includes three segments called complementarity-determining regions (CDRs) or hypervariable regions and a more highly conserved portions of variable domains are called the framework region (FR). The variable domains of heavy and light chains each includes four FR regions, largely adopting a β-sheet configuration, connected by three CDRs, which form loops connecting, and in some cases forming part of the β-sheet structure. The CDRs in each chain are held together in close proximity by the FRs and, with the CDRs from the other chain, contribute to the formation of the antigen-binding domain or targeting domain of antibodies (scc Kabat et al., NIH Publ. No. 91-3242, Vol. I, pages 647-669 [1991] ). The constant domains are not involved directly in binding an antibody to an antigen, but exhibit various effector functions, such as participation of the antibody in antibody dependent cellular cytotoxicity.

Antibodies can be cleaved experimentally with the proteolytic enzyme papain, which causes each of the heavy chains to break, producing three separate antibody fragments. The two units that consist of a light chain and a fragment of the heavy chain approximately equal in mass to the light chain are called the Fab fragments (i.e., the “antigen binding” fragments). The third unit, consisting of two equal segments of the heavy chain, is called the Fc fragment. The Fc fragment is typically not involved in antigen-antibody binding but is important in later processes involved in ridding the body of the antigen. As used herein, “antibody fragments” include a portion of an intact antibody, preferably the antigen binding or variable region of the intact antibody. Examples of antibody fragments include Fab, Fab′ and F(ab′)2, Fc fragments or Fc-fusion products, single-chain Fvs (scFv), disulfide-linked Fvs (sdfv) and fragments including either a VL or VH domain; diabodies, tribodies and the like (Zapata et al. Protein Eng. 8 (10): 1057-1062 [1995]).

The Fab fragment contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

The Fc region of an antibody is the tail region of an antibody that interacts with cell surface receptors and some proteins of the complement system. This property allows antibodies to activate the immune system. In IgG, IgA and IgD antibody isotypes, the Fc region is composed of two identical protein fragments, derived from the second and third constant domains of the antibody's two heavy chains; IgM and IgE Fc regions contain three heavy chain constant domains (CH domains 2-4) in each polypeptide chain. The Fc regions of IgGs bear a highly conserved N-glycosylation site. Glycosylation of the Fc fragment is essential for Fc receptor-mediated activity. The N-glycans attached to this site are predominantly core-fucosylated diantennary structures of the complex type. In addition, small amounts of these N-glycans also bear bisecting GlcNAc and α-2,6 linked sialic acid residues.

Fc-Fusion proteins (also known as Fc chimeric fusion protein, Fc-Ig, Ig-based Chimeric Fusion protein and Fc-tag protein) are composed of the Fc domain of IgG genetically linked to a peptide or protein of interest. Fc-Fusion proteins have become valuable reagents for in vivo and in vitro research. The Fc-fused binding partner can range from a single peptide, a ligand that activates upon binding with a cell surface receptor, signaling molecules, the extracellular domain of a receptor that is activated upon dimerization or as a bait protein that is used to identify binding partners in a protein microarray. One of the most valuable features of the Fc domain in vivo, is it can dramatically prolong the plasma half-life of the protein of interest, which for bio-therapeutic drugs, results in an improved therapeutic efficacy; an attribute that has made Fc-Fusion proteins attractive bio-therapeutic agents. The Fc fusion protein may be part of a pharmaceutical composition including an Fc fusion protein and a pharmaceutically acceptable carrier excipients or carrier. Pharmaceutically acceptable carriers, excipients or stabilizers are well known in the art (Remington's Pharmaceutical Sciences, 16th edition, Osol, A. Ed. (1980)). Acceptable carriers, excipients, or stabilizers are nontoxic to recipients at the dosages and concentrations employed, and may include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (for example, Zn-protein complexes); and/or non-ionic surfactants such as TWEEN™, PLURONICS™ or polyethylene glycol (PEG).

“Fv” is the minimum antibody fragment which contains a complete antigen-recognition and-binding site. This region consists of a dimer of one heavy-and one light-chain variable domain in tight, non-covalent association. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

“Single-chain Fv” or “sFv” antibody fragments comprise the VH and VL domains of antibody, wherein these domains are present in a single polypeptide chain. Preferably, the Fv polypeptide further comprises a polypeptide linker between the VH and VL domains which enables the sFv to form the desired structure for antigen binding. For a review of sFv sec Pluckthun in The Pharmacology of Monoclonal Antibodies, vol. 113, Rosenburg and Moore cds., Springer-Verlag, New York, pp. 269-315 (1994).

Various techniques have been developed for the production of antibody fragments. Traditionally, these fragments were derived via proteolytic digestion of intact antibodies (sec, e.g., Morimoto et al., Journal of Biochemical and Biophysical Methods 24:107-117 (1992) and Brennan et al., Science, 229:81 [1985] ). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′2 fragments (Carter et al., Bio/Technology 10:163-167 [1992] ). According to another approach, F(ab′)2 fragments can be isolated directly from recombinant host cell culture. Other techniques for the production of antibody fragments will be apparent to the skilled practitioner. In other embodiments, the antibody of choice is a single chain Fv fragment (scFv). Sec WO 93/16185.

In various aspects, the nucleic acid sequences provided herein encode a light chain and a heavy chain that bind specifically to a PD-L1 protein.

Programmed cell death protein 1, also known as PD-1 and CD279 (cluster of differentiation 279), is a cell surface receptor that plays an important role in down-regulating the immune system and promoting self-tolerance by suppressing T cell inflammatory activity. PD-1 is an immune checkpoint and guards against autoimmunity through a dual mechanism of promoting apoptosis (programmed cell death) in antigen-specific T-cells in lymph nodes while simultaneously reducing apoptosis in regulatory T cells (anti-inflammatory, suppressive T cells). PD-1 has two ligands, PD-L1 and PD-L2, which are members of the B7 family. PD-L1 protein is upregulated on macrophages and dendritic cells (DC) in response to LPS and GM-CSF treatment, and on T cells and B cells upon TCR and B cell receptor signaling, whereas in resting mice, PD-L1 mRNA can be detected in the heart, lung, thymus, spleen, and kidney. PD-1 is a member of the extended CD28/CTLA-4 family of T cell regulators. PD-1 is expressed on the surface of activated T cells, B cells, and macrophages, suggesting that compared to CTLA-4, PD-1 more broadly negatively regulates immune responses.

PD-1 has two ligands, PD-L1 and PD-L2, which are members of the B7 family. PD-L1 binds to its receptor, PD-1, found on activated T cells, B cells, and myeloid cells, to modulate activation or inhibition. The affinity between PD-L1 and PD-1, as defined by the dissociation constant Kd, is 770 nM. PD-L1 also has an appreciable affinity for the costimulatory molecule CD80 (B7-1), but not CD86 (B7-2). Engagement of PD-L1 with its receptor PD-1 on T cells delivers a signal that inhibits TCR-mediated activation of IL-2 production and T cell proliferation. The mechanism involves inhibition of ZAP70 phosphorylation and its association with CD3ζ. PD-1 signaling attenuates PKC-θ activation loop phosphorylation (resulting from TCR signaling), necessary for the activation of transcription factors NF-κB and AP-1, and for production of IL-2. PD-L1 binding to PD-1 also contributes to ligand-induced TCR down-modulation during antigen presentation to naive T cells, by inducing the up-regulation of the E3 ubiquitin ligase CBL-b. Upon IFN-γstimulation, PD-L1 is expressed on T cells, NK cells, macrophages, myeloid DCs, B cells, epithelial cells, and vascular endothelial cells. The PD-L1 gene promoter region has a response element to IRF-1, the interferon regulatory factor.

PD-L1 plays a major role in suppressing the adaptive arm of immune system during particular events such as pregnancy, tissue allografts, autoimmune disease and other disease states such as hepatitis. In normal conditions, the adaptive immune system reacts to antigens that are associated with immune system activation by exogenous or endogenous danger signals. In turn, clonal expansion of antigen-specific CD8+ T cells and/or CD4+ helper cells is propagated. The binding of PD-L1 to the inhibitory checkpoint molecule PD-1 transmits an inhibitory signal based on interaction with phosphatases (SHP-1 or SHP-2) via Immunoreceptor Tyrosine-Based Switch Motif (ITSM). This reduces the proliferation of antigen-specific T-cells in lymph nodes, while simultaneously reducing apoptosis in regulatory T cells (anti-inflammatory, suppressive T cells)—further mediated by a lower regulation of the gene Bcl-2.

By overexpressing PD-L1 (constitutively or by inducing its expression) and/or inhibit PD-L1 degradation, cancer cells develop an immune resistance mechanism in response to the endogenous immune anti-tumor activity and escape anti-tumor immunity. PD-L1 is overexpressed on tumor cells and on non-transformed cells in the tumor microenvironment, leading to the depletion of the tumor microenvironment of cytotoxic T cells, to tumor cells survival and proliferation, and to cancer progression.

“PD-L1 targeting peptide” or “PD-L1 targeting protein” is meant to refer to any peptide or polypeptide (including protein and fusion protein) that can specifically bind to PD-L1. The PD-L1 targeting peptide can be an antibody, an antibody fragment, and the like, having specific binding to one or more target polypeptide, including PD-L1. In some aspects, the polypeptide encodes the light chain and the heavy chain of a PD-L1 targeting peptide. In one aspect, the nucleic acid sequence of SEQ ID NO:13 can encode the light chain of a PD-L1 targeting peptide, having the amino acid sequence as set forth in SEQ ID NO:6. In another aspect, the nucleic acid sequence of SEQ ID NO: 14 can encode the heavy chain of a PD-L1 targeting peptide, having the amino acid sequence as set forth in SEQ ID NO:7.

In an additional embodiment, the invention provides a fusion protein including the amino acid sequence set forth in SEQ ID NO:6 and 7, operably linked to each other in either orientation.

The terms “fusion molecule” and “fusion protein” are used interchangeably and are meant to refer to a biologically active polypeptide, with or without a further effector molecule, usually a protein or peptide sequence covalently linked (i.e. fused) by recombinant, chemical or other suitable method. If desired, the fusion molecule can be used at one or several sites through a peptide linker sequence. Alternatively, the peptide linker may be used to assist in construction of the fusion molecule. Specifically, preferred fusion molecules are fusion proteins. Generally fusion molecule also can include conjugate molecules.

By “operably linked” to one another, it is meant that there is a direct or indirect covalent linking between the peptides composing the fusion protein. Thus, two domains that are operably linked may be directly covalently coupled to one another. Conversely, the two operably linked domains may be connected by mutual covalent linking to an intervening moiety (e.g., and flanking sequence). Two domains may be considered operably linked if, for example, they are separated by the third domain, with or without one or more intervening flanking sequences.

Methods for attaching two individual elements usually require the use of a linker. The term “linker” as used herein refers any bond, small molecule, or other vehicle which allows the substrate and the active agent to be targeted to the same area, tissue, or cell, for example by physically linking the individual portions of the conjugate. A linker can be any chemical moiety that is capable of linking a compound, usually a drug, to a cell-binding agent in a stable, covalent manner.

The fusion proteins provided herein can for example include the amino acid sequences set forth in SEQ ID NOs: 6 and 7, operably linked to each other in either orientation. For example, the fusion protein can include the amino acid sequence set forth in SEQ ID NO:6 at a C-terminal of the fusion protein and the amino acid sequence set forth in SEQ ID NO:7 at a N-terminal of the fusion protein; or the fusion protein can include the amino acid sequence set forth in SEQ ID NO:6 at a N-terminal of the fusion protein and the amino acid sequence set forth in SEQ ID NO:7 at a C-terminal of the fusion protein. The orientation of the amino acid sequences in the fusion protein do not alter the binding-specificity of the fusion protein to its target (i.e., PD-L1 targeting fusion protein).

The light chain and the heavy chain of the B7-H3 targeting peptide can be operably linked to one another in either orientation without affecting the binding specificity or sensitivity of the targeting peptide. In one aspect, the protein includes SEQ ID NO:6 and 7, in direct linkage between the C-terminus of SEQ ID NO:6 and the N-terminus of SEQ ID NO:7. In another aspect, the protein includes SEQ ID NO:7 and 6, in direct linkage between the C-terminus of SEQ ID NO:7 and the N-terminus of SEQ ID NO:6.

The fusion protein provided herein can include additional protein domain, such as additional targeting domain to provide the fusion protein with specific binding to one or more target polypeptide. For example, the fusion protein can be a tri-specific killer engager (TrikE) molecule including the PD-L1 targeting peptide as the targeting domain.

NK cells are cytotoxic lymphocytes of the innate immune system capable of immune surveillance. Like cytotoxic T cells, NK cells deliver a store of membrane penetrating and apoptosis-inducing granzyme and perforin granules. Unlike T cells, NK cells do not require antigen priming and recognize targets by engaging activating receptors in the absence of MHC recognition. NK cells express CD16, an activation receptor that binds to the Fc portion of IgG antibodies and is involved in antibody-dependent cell-mediated cytotoxicity (ADCC). NK cells are regulated by IL-15, which can induce increased antigen-dependent cytotoxicity, lymphokine-activated killer activity, and/or mediate interferon (IFN), tumor-necrosis factor (TNF) and/or granulocyte-macrophage colony-stimulating factor (GM-CSF) responses. All of these IL-15-activated functions contribute to improved cancer defense.

Therapeutically, adoptive transfer of NK cells can, for example, induce remission in patients with refractory acute myeloid leukemia (AML) when combined with lymphodepleting chemotherapy and IL-2 to stimulate survival and in vivo expansion of NK cells. This therapy can be limited by lack of antigen specificity and IL-2-mediated induction of regulatory T (Treg) cells that suppress NK cell proliferation and function. Generating a reagent that drives NK cell antigen specificity, expansion, and/or persistence, while bypassing the negative effects of Treg inhibition, can enhance NK-cell-based immunotherapies.

Tri-specific killer engager molecule are targeting fusion protein including two domains capable of driving NK-cell-mediated killing of tumor cells (e.g., CD33+ tumor cells and/or EpCAM+tumor cells) and an intramolecular NK activating domain capable of generating an NK cell self-sustaining signal can drive NK cell proliferation and/or enhance NK-cell-driven cytotoxicity against, for example, HL-60 targets, cancer cells, or cancer cell-derived cell lines.

NK cells are responsive to a variety of cytokines including, for example, IL-15, which is involved in NK cell homeostasis, proliferation, survival, activation, and/or development. IL-15 and IL-2 share several signaling components, including the IL-2/IL-15Rβ (CD122) and the common gamma chain (CD132). Unlike IL-2, IL-15 does not stimulate Tregs, allowing for NK cell activation while bypassing Treg inhibition of the immune response. Besides promoting NK cell homeostasis and proliferation, IL-15 can rescue NK cell functional defects that can occur in the post-transplant setting. IL-15 also can stimulate CD8+ T cell function, further enhancing its immunotherapeutic potential. In addition, based on pre-clinical studies, toxicity profiles of IL-15 may be more favorable than IL-2 at low doses. IL-15 plays a role in NK cell development homeostasis, proliferation, survival, and activation. IL-15 and IL-2 share several signaling components including the IL-2/IL-15Rβ (CD122) and the common gamma chain (CD132). IL-15 also activates NK cells, and can restore functional defects in engrafting NK cells after hematopoietic stem cell transplantation (HSCT).

The fusion protein provided herein can be a TriKE molecule including one or more NK cell engager domains (e.g., CD16, CD16+CD2, CD16+DNAM, CD16+NKp46), one or more targeting domains (that target, e.g., a tumor cell or virally-infected cell, such as the PD-L1 targeting peptide described herein), and one or more cytokine NK activating domains (e.g., IL-15, IL-12, IL-18, IL-21, or other NK cell enhancing cytokine, chemokine, and/or activating molecule), with each domain operably linked to the other domains.

For example, the fusion protein described herein can be a TriKE molecule including a CD16 NK cell engager domain, such as the CD16 domain having the amino acid sequence set forth in SEQ ID NO:2 or 23; a PD-L1 targeting fusion protein domain, such as the PD-L1 fusion protein having the amino acid sequences set forth in SEQ ID NOs: 6 and 7; and a IL-15 cytokine NK activating domain, such as the IL-15 having the amino acid sequence set forth in SEQ ID NO:4, 21 or 22.

The different protein domains of the TriKE molecules can be in operable linkage with one another. For example, linkers can be used to covalently attached the protein domains of the TriKE molecule to one another.

The elements of a fusion protein can be in assembled operable linkage with one another using one or more linkers. Linkers can be susceptible to or be substantially resistant to acid-induced cleavage, light-induced cleavage, peptidase-induced cleavage, esterase-induced cleavage and disulfide bond cleavage at conditions under which the compound or the antibody remains active. Linkers are classified upon their chemical motifs, well known in the art, including disulfide groups, hydrazine or peptides (cleavable), or thioester groups (non-cleavable). Linkers also include charged linkers, and hydrophilic forms thereof as known in the art.

Suitable linker for the fusion of two or more protein or protein domains can include natural linkers, and empirical linkers. Natural linkers are derived from multi-domain proteins, which are naturally present between protein domains. Natural linkers can have several properties depending or their such as length, hydrophobicity, amino acid residues, and secondary structure, which can impact the fusion protein in different way.

The studies of linkers in natural multi-domain proteins have led to the generation of many empirical linkers with various sequences and conformations for the construction of recombinant fusion proteins. Empirical linkers can be classified in three types: flexible linkers, rigid linkers, and cleavable linkers. Flexible linkers can provide a certain degree of movement or interaction at the joined domains. They are generally composed of small, non-polar (e.g. Gly) or polar (e.g. Ser or Thr) amino acids, which provides flexibility, and allows for mobility of the connecting functional domains. Rigid linkers can successfully keep a fixed distance between the domains to maintain their independent functions, which can provide efficient separation of the protein domains or sufficient reduction of their interference with each other. Cleavable linkers can allow the release of functional domains in vivo. By taking advantage of unique in vivo processes, they can be cleaved under specific conditions such as the presence of reducing reagents or proteases. This type of linker can reduce steric hindrance, improve bioactivity, or achieve independent actions/metabolism of individual domains of recombinant fusion proteins after linker cleavage.

Non limiting examples of linker include linkers having the amino acid sequences set forth in SEQ ID NOs: 3, 5, 16 and 17.

In one aspect, SEQ ID NO:2 or 23 and 4, 21 or 22 are linked by SEQ ID NO:3 or SEQ ID NO:16. In another aspect, SEQ ID NO:4, 17 or 18 and 6 or 7 are linked by SEQ ID NO: 5 or SEQ ID NO:17. In other aspects, SEQ ID NO:6 and 7 are in operable linkage in either orientation.

In a further embodiment, the invention provides a fusion protein including the sequence set forth in SEQ ID NO:1 or 15 and sequences having 90% or greater identity to SEQ ID NO: 1 or 15.

In one embodiment, the present invention provides a fusion protein including in operably linkage, SEQ ID NO:2 or 23; 4, 21 or 22; 6 and 7 or 7 and 6.

The fusion protein described herein can include a wild-type (wt) IL-15 or mutant IL-15 cytokine NK activating domain. Mutant IL-15 can for example include IL-15 including a substitution of the N72 amino acid. Non-limiting examples of N72 substitutions include N72A and N72D mutations.

In some aspects, SEQ ID NO:4 has an N72 substitution. In various aspects, the N72 mutation is N72A or N72D and the protein is set forth in SEQ ID NO:21 or 22, respectively.

In one embodiment, the invention provides a fusion protein including SEQ ID NO: 23, SEQ ID NO:21 or 22 and SEQ ID NO:6 and 7 in either orientation. In one aspect, SEQ ID NO: 23 is operably linked to SEQ ID NO:21 or 22 by a linker of SEQ ID NO:3 or 16. In another aspect, SEQ ID NO:21 or 22 is operably linked to SEQ 6 and 7, in either orientation by a linker of SEQ ID NO:5 or 17.

The fusion protein can include in operable linkage a camelid or a human CD16 NK cell engager domain (SEQ ID NO:2 or 23, respectively), a wt or a mutant IL-15 cytokine NK activating domain (SEQ ID NO:4, 21 or 22), and a light chain and a heavy chain of an of a PD-L1 targeting peptide (SEQ ID NO:6 and 7, respectively). The CD16 NK cell engager domain can be linked to IL-15 cytokine NK activating domain by a linker having an amino acid sequence set forth in SEQ ID NO:3 or 16. The IL-15 cytokine NK activating domain can be linked to the PD-L1 targeting peptide by a linker having an amino acid sequence set forth in SEQ ID NO:5 or 17. The IL-15 cytokine NK activating domain can be linked to the heavy chain of the PD-L1 targeting peptide (linked to the light chain), or to the light chain of the B7-H3 targeting peptide (linked to the heavy chain).

For example, the fusion protein can include, in operable linkage, from an N-terminus to a C-terminus, SEQ ID NOs: 2, 4, 6 and 7; SEQ ID NOs: 2, 4, 7 and 6; SEQ ID NOs: 23, 21, 6 and 7; SEQ ID NOs: 23, 21, 7 and 6; SEQ ID NOs: 23, 22, 6 and 7; or SEQ ID NOs: 23, 22, 7 and 6.

Specifically, the fusion protein can include, in operable linkage, from a N-terminus to a C-terminus, SEQ ID NOs: 2, 3, 4, 5, 6 and 7; SEQ ID NOs: 2, 3, 4, 17, 6 and 7; SEQ ID NOs: 2, 16, 4, 5, 6 and 7; SEQ ID NOs: 2, 16, 4, 17, 6 and 7; SEQ ID NOs: 2, 3, 4, 5, 7 and 6; SEQ ID NOs: 2, 3, 4, 17, 7 and 6; SEQ ID NOs: 2, 16, 4, 5, 7 and 6; or SEQ ID NOs: 2, 16, 4, 17, 7 and 6.

In other aspects, the fusion protein can include, in operable linkage, from a N-terminus to a C-terminus, SEQ ID NOs: 23, 3, 21, 5, 6 and 7; SEQ ID NOs: 23, 3, 21, 17, 6 and 7; SEQ ID NOs: 23, 16, 21, 5, 6 and 7; SEQ ID NOs: 23, 16, 21, 17, 6 and 7; SEQ ID NOs: 23, 3, 21, 5, 7 and 6; SEQ ID NOs: 23, 3, 21, 17, 7 and 6; SEQ ID NOs: 23, 16, 21, 5, 7 and 6; SEQ ID NOs: 23, 16, 21, 17, 7 and 6; SEQ ID NOs: 23, 3, 22, 5, 6 and 7; SEQ ID NOs: 23, 3, 22, 17, 6 and 7; SEQ ID NOs: 23, 16, 22, 5, 6 and 7; SEQ ID NOs: 23, 16, 22, 17, 6 and 7; SEQ ID NOs: 23, 3, 22, 5, 7 and 6; SEQ ID NOs: 23, 3, 22, 17, 7 and 6; SEQ ID NOs: 23, 16, 22, 5, 7 and 6; or SEQ ID NOs: 23, 16, 22, 17, 7 and 6.

In some aspects, the fusion protein further includes a half-life extending (HLE) molecule.

The circulatory half-life of targeting proteins such as IgG immunoglobulins can be regulated by the affinity of the Fc region for the neonatal Fc receptor (FcRn). The second general category of effector functions include those that operate after an immunoglobulin binds an antigen. In the case of IgG, these functions involve the participation of the complement cascade or Fc gamma receptor (FcγR)-bearing cells. Binding of the Fc region to an FcγR causes certain immune effects, for example, endocytosis of immune complexes, engulfment and destruction of immunoglobulin-coated particles or microorganisms (also called antibody-dependent phagocytosis, or ADCP), clearance of immune complexes, lysis of immunoglobulin-coated target cells by killer cells (called antibody-dependent cell-mediated cytotoxicity, or ADCC), release of inflammatory mediators, regulation of immune system cell activation, and regulation of immunoglobulin production. Certain engineered binding polypeptides (e.g., antibody variants (e.g., scFvs) or antibody fragments (e.g., Fab fragments)), while benefiting from their smaller molecular size and/or monovalency, also suffer several disadvantages attributable to the absence of a functional Fc region. For example, Fab fragments have short half-lives in vivo because they lack the Fc region that is required for FcRn binding and are rapidly filtered out of the blood by the kidneys owing to their small size.

Engineered targeting polypeptides, such as the fusion proteins described herein, can exhibit decreased binding to FcRn when compared to native binding polypeptides and, therefore, have decreased half-life in serum. Fc variants with improved affinity for FcRn are anticipated to have longer serum half-lives, and such molecules have useful applications in methods of treating mammals where long half-life of the administered polypeptide is desired, e.g., to treat a chronic disease or disorder. In contrast, Fc variants with decreased FcRn binding affinity are expected to have shorter half-lives, and such molecules are also useful, for example, for administration to a mammal where a shortened circulation time may be advantageous, e.g. for in vivo diagnostic imaging or in situations where the starting polypeptide has toxic side effects when present in the circulation for prolonged periods.

In some aspects, the fusion protein further includes a half-life extending (HLE) molecule.

The circulatory half-life of targeting proteins such as IgG immunoglobulins can be regulated by the affinity of the Fc region for the neonatal Fc receptor (FcRn). The second general category of effector functions include those that operate after an immunoglobulin binds an antigen. In the case of IgG, these functions involve the participation of the complement cascade or Fc gamma receptor (FcγR)-bearing cells. Binding of the Fc region to an FcγR causes certain immune effects, for example, endocytosis of immune complexes, engulfment and destruction of immunoglobulin-coated particles or microorganisms (also called antibody-dependent phagocytosis, or ADCP), clearance of immune complexes, lysis of immunoglobulin-coated target cells by killer cells (called antibody-dependent cell-mediated cytotoxicity, or ADCC), release of inflammatory mediators, regulation of immune system cell activation, and regulation of immunoglobulin production. Certain engineered binding polypeptides (e.g., antibody variants (e.g., scFvs) or antibody fragments (e.g., Fab fragments)), while benefiting from their smaller molecular size and/or monovalency, also suffer several disadvantages attributable to the absence of a functional Fc region. For example, Fab fragments have short half-lives in vivo because they lack the Fc region that is required for FcRn binding and are rapidly filtered out of the blood by the kidneys owing to their small size.

Engineered targeting polypeptides, such as the fusion proteins described herein, can exhibit decreased binding to FcRn when compared to native binding polypeptides and, therefore, have decreased half-life in vivo. Fc variants with improved affinity for FcRn can have longer serum half-lives, and such molecules have useful applications in methods of treating mammals where long half-life of the administered polypeptide is desired, e.g., to treat a chronic disease or disorder. In contrast, Fc variants with decreased FcRn binding affinity have shorter half-lives, and such molecules are also useful, for example, for administration to a mammal where a shortened circulation time may be advantageous, e.g. for in vivo diagnostic imaging or in situations where the starting polypeptide has toxic side effects when present in the circulation for prolonged periods.

The fusion proteins described herein can include a half-life extending (HLE) molecule to extend their half-life in vivo upon administration to a subject.

As used herein, the term “half-life” refers to a biological half-life of a particular targeting polypeptide in vivo. Half-life may be represented by the time required for half the quantity administered to a subject to be cleared from the circulation and/or other tissues in the animal. When a clearance curve of a targeting polypeptide is constructed as a function of time, the curve is usually biphasic with a rapid α-phase and longer β-phase. The α-phase typically represents an equilibration of the administered targeting polypeptide between the intra- and extra-vascular space and is, in part, determined by the size of the polypeptide. The β-phase typically represents the catabolism of the targeting polypeptide in the intravascular space. Therefore, the term half-life as used herein preferably refers to the half-life of the targeting polypeptide in the β-phase. The typical β phase half-life of a human antibody in humans is 21 days.

An increased half-life is generally useful in in vivo applications of immunoglobulins, especially antibodies and most especially antibody fragments of small size. Approaches described in the art to achieve such effect comprise the fusion of the small bispecific antibody construct to larger proteins, which preferably do not interfere with the therapeutic effect of the protein construct. Examples for such further developments of bispecific T cell engagers are described in US 2017/0218078A1, which provides half-life extending formats (HLE formats) of bispecific T cell engaging molecules comprising a first domain binding to a target cell surface antigen, a second domain binding to an extracellular epitope of the human and/or the Macaca CD3ϵ chain and a third domain, which is the specific Fc modality (the HLE molecule).

As used herein, the terms “half-life extending molecule”, “HLE sequence” and the like are meant to refer to any molecule, such as a protein or polypeptide that can be linked or fused to a polypeptide of interest to increase or extend its half-life in vivo. Specifically, a HLE sequence generally includes a Fc region or scFc region of an immunoglobulin.

As used herein, the term “Fc region” refers to the portion of a native immunoglobulin formed by the respective Fc domains (or Fc moieties) of its two heavy chains. A native Fc region is homodimeric. In contrast, the term “genetically-fused Fc region” or “single-chain Fc region” (scFc region), as used herein, refers to a synthetic Fc region comprised of Fc domains (or Fc moieties) genetically linked within a single polypeptide chain (i.e., encoded in a single contiguous genetic sequence). Accordingly, a genetically fused Fc region (i.e., a scFc region) is monomeric.

The term “Fc domain” refers to the portion of a single immunoglobulin heavy chain beginning in the hinge region just upstream of the papain cleavage site (i.e. residue 216 in IgG, taking the first residue of heavy chain constant region to be 114) and ending at the C-terminus of the antibody. Accordingly, a complete Fc domain comprises at least a hinge domain, a CH2 domain, and a CH3 domain.

The scFc region described herein includes at least two Fc domain which are genetically fused via a linker polypeptide (e.g., an Fc connecting peptide) interposed between said Fc moieties. The scFc region can include two identical Fc moieties or can include two non-identical Fc moieties.

Non-limiting examples of Fc domain that can be used for the preparation of a HLE molecule (alone or in combination with another Fc domain through a linker polypeptide) that can be incorporated in any of the fusion proteins described herein include any of the polypeptides having an amino acid including any one of SEQ ID NOs: 30-37.

Non-limiting examples of linker polypeptide that can be used for the preparation of a scFc region that can be used for the preparation of a HLE molecule include any of the polypeptides having an amino acid including any one of SEQ ID NOs: 38-39.

The HLE molecules described herein can include a Fc domain having an amino acid including any one of SEQ ID NOs: 30-37, or a scFc region including a first Fc domain having an amino acid comprising any one of SEQ ID NOs: 30-37 fused to a second Fc domain having an amino acid comprising any one of SEQ ID NOs: 30-37, through a linker having an amino acid including any one of SEQ ID NOs: 38-39. For example, the HLE molecule can include any one of SEQ ID NOs: 25-29.

In an additional embodiment, the invention provides an isolated nucleic acid sequence encoding any of the fusion proteins described herein.

The fusion proteins described herein, such as the TriKE fusion protein including a CD16 NK cell engager domain, such as the CD16 domain having the amino acid sequence set forth in SEQ ID NO:2; a PD-L1 targeting fusion protein domain, such as the PD-L1 fusion protein having the amino acid sequences set forth in SEQ ID NOs: 6 and 7; and a IL-15 cytokine NK activating domain, such as the IL-15 having the amino acid sequence set forth in SEQ ID NO: 4, in operable linkage, and as set forth in SEQ ID NO: 1 can be encoded by a nucleic acid sequence. In one aspect, the sequence is SEQ ID NO:8 or 18 or sequences having 90% or more sequence identity thereto.

In another embodiment, the invention provides a method of treating cancer in a subject including administering to the subject any of the fusion protein described herein, thereby treating the cancer.

The term “subject” as used herein refers to any individual or patient to which the subject methods are performed. Generally, the subject is human, although as will be appreciated by those in the art, the subject may be an animal. Thus other animals, including vertebrate such as rodents (including mice, rats, hamsters and guinea pigs), cats, dogs, rabbits, farm animals including cows, horses, goats, sheep, pigs, chickens, etc., and primates (including monkeys, chimpanzees, orangutans and gorillas) are included within the definition of subject.

The term “treatment” is used interchangeably herein with the term “therapeutic method” and refers to both 1) therapeutic treatments or measures that cure, slow down, lessen symptoms of, and/or halt progression of a diagnosed pathologic conditions or disorder, and 2) and prophylactic/preventative measures. Those in need of treatment may include individuals already having a particular medical disorder as well as those who may ultimately acquire the disorder (i.e., those needing preventive measures).

The terms “therapeutically effective amount”, “effective dose,” “therapeutically effective dose”, “effective amount,” or the like refer to that amount of the subject compound that will elicit the biological or medical response of a tissue, system, animal or human that is being sought by the researcher, veterinarian, medical doctor or other clinician. Generally, the response is either amelioration of symptoms in a patient or a desired biological outcome. Such amount should be sufficient to treat cancer. The effective amount can be determined as described herein.

The terms “administration of” and or “administering” should be understood to mean providing a pharmaceutical composition in a therapeutically effective amount to the subject in need of treatment. Administration routes can be enteral, topical or parenteral. As such, administration routes include but are not limited to intracutaneous, subcutaneous, intravenous, intraperitoneal, intraarterial, intrathecal, intracapsular, intraorbital, intracardiac, intradermal, transdermal, transtracheal, subcuticular, intraarticulare, subcapsular, subarachnoid, intraspinal and intrasternal, oral, sublingual buccal, rectal, vaginal, nasal ocular administrations, as well infusion, inhalation, and nebulization. The phrases “parenteral administration” and “administered parenterally” as used herein means modes of administration other than enteral and topical administration.

The fusion proteins described herein can be formulated in pharmaceutical compositions comprising the fusion protein and a pharmaceutically acceptable carrier. By “pharmaceutically acceptable” it is meant the carrier, diluent or excipient must be compatible with the other ingredients of the formulation and not deleterious to the recipient thereof. Examples of carrier include, but are not limited to, liposome, nanoparticles, ointment, micelles, microsphere, microparticle, cream, emulsion, and gel. Examples of excipient include, but are not limited to, anti-adherents such as magnesium stearate, binders such as saccharides and their derivatives (sucrose, lactose, starches, cellulose, sugar alcohols and the like) protein like gelatin and synthetic polymers, lubricants such as talc and silica, and preservatives such as antioxidants, vitamin A, vitamin E, vitamin C, retinyl palmitate, selenium, cysteine, methionine, citric acid, sodium sulfate and parabens. Examples of diluent include, but are not limited to, water, alcohol, saline solution, glycol, mineral oil and dimethyl sulfoxide (DMSO).

Pharmaceutical compositions can be administered in a variety of unit dosage forms depending upon the method of administration. Suitable unit dosage forms, include, but are not limited to powders, tablets, pills, capsules, lozenges, suppositories, patches, nasal sprays, injectables, implantable sustained-release formulations, lipid complexes, etc.

The methods described herein are directed to the treatment of cancer. The term “cancer” refers to a group of diseases characterized by abnormal and uncontrolled cell proliferation starting at one site (primary site) with the potential to invade and to spread to others sites (secondary sites, metastases) which differentiates cancer (malignant tumor) from benign tumor. Virtually all the organs can be affected, leading to more than 100 types of cancer that can affect humans. Cancers can result from many causes including genetic predisposition, viral infection, exposure to ionizing radiation, exposure environmental pollutant, tobacco and/or alcohol use, obesity, poor diet, lack of physical activity or any combination thereof. As used herein, “neoplasm” or “tumor” including grammatical variations thereof, means new and abnormal growth of tissue, which may be benign or cancerous. In a related aspect, the neoplasm is indicative of a neoplastic disease or disorder, including but not limited, to various cancers. For example, such cancers can include prostate, pancreatic, biliary, colon, rectal, liver, kidney, lung, testicular, breast, ovarian, brain, and head and neck cancers, melanoma, sarcoma, multiple myeloma, leukemia, lymphoma, and the like.

Exemplary cancers described by the national cancer institute include: Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood: Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma. Childhood Brain Stem; Glioma. Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's; Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplasia Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood', Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland's Cancer, Childhood; Sarcoma, Ewing's Family of Tumors; Sarcoma, Kaposi's; Sarcoma (Osteosarcoma) Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor.

In one aspect, the cancer is selected from non-small lung cancer, cutaneous squamous cell carcinoma, pancreatic cancer, primary hepatocellular carcinoma, colorectal carcinoma, clear cell renal carcinoma, prostate cancer, cervical cancer, ovarian cancer, melanoma, brain cancer, leukemia, lymphoma, myeloma, head and neck cancer or breast cancer.

In some aspects, administration of the fusion proteins described herein can be in combination with one or more additional therapeutic agents. The phrases “combination therapy”, “combined with” and the like refer to the use of more than one medication or treatment simultaneously to increase the response. The fusion proteins of the present invention and the pharmaceutical composition thereof might for example be used in combination with other drugs or treatment in use to treat cancer. Specifically, the administration of the fusion proteins to a subject can be in combination with a chemotherapeutic agent, surgery, radiotherapy, or a combination thereof. Such therapies can be administered prior to, simultaneously with, or following administration of the composition of the present invention.

The term “chemotherapeutic agent” as used herein refers to any therapeutic agent used to treat cancer. Examples of chemotherapeutic agents include, but are not limited to, Actinomycin, Azacitidine, Azathioprine, Bleomycin, Bortezomib, Carboplatin, Capecitabine, Cisplatin, Chlorambucil, Cyclophosphamide, Cytarabine, Daunorubicin, Docetaxel, Doxifluridine, Doxorubicin, Epirubicin, Epothilone, Etoposide, Fluorouracil, Gemcitabine, Hydroxyurca, Idarubicin, Imatinib, Irinotecan, Mechlorethamine, Mercaptopurine, Methotrexate, Mitoxantrone, Oxaliplatin, Paclitaxel, Pemetrexed, Teniposide, Tioguanine, Topotecan, Valrubicin, Vinblastine, Vincristine, Vindesine, Vinorelbine, panitumamab, Erbitux (cetuximab), matuzumab, IMC-IIF 8, TheraCIM hR3, denosumab, Avastin (bevacizumab), Humira (adalimumab), Herceptin (trastuzumab), Remicade (infliximab), rituximab, Synagis (palivizumab), Mylotarg (gemtuzumab oxogamicin), Raptiva (cfalizumab), Tysabri (natalizumab), Zenapax (dacliximab), NeutroSpec (Technetium (99mTc) fanolesomab), tocilizumab, ProstaScint (Indium-III labeled Capromab Pendetide), Bexxar (tositumomab), Zevalin (ibritumomab tiuxetan (IDEC-Y2B8) conjugated to yttrium 90), Xolair (omalizumab), MabThera (Rituximab), RcoPro (abciximab), MabCampath (alemtuzumab), Simulect (basiliximab), LeukoScan (sulesomab), CEA-Scan (arcitumomab), Verluma (nofctumomab), Panorex (Edrecolomab), alemtuzumab, CDP 870, natalizumab Gilotrif (afatinib), Lynparza (olaparib), Perjeta (pertuzumab), Otdivo (nivolumab), Bosulif (bosutinib), Cabometyx (cabozantinib), Ogivri (trastuzumab-dkst), Sutent (sunitinib malate), Adcetris (brentuximab vedotin), Alecensa (alectinib), Calquence (acalabrutinib), Yescarta (ciloleucel), Verzenio (abemaciclib), Keytruda (pembrolizumab), Aliqopa (copanlisib), Nerlynx (neratinib), Imfinzi (durvalumab), Darzalex (daratumumab), Tecentriq (atezolizumab), and Tarceva (erlotinib). Examples of immunotherapeutic agent include, but are not limited to, interleukins (Il-2, Il-7, Il-12), cytokines (Interferons, G-CSF, imiquimod), chemokines (CCL3, CC126, CXCL7), immunomodulatory imide drugs (thalidomide and its analogues).

In some aspects, an immune checkpoint inhibitor is further administered to the subject.

Immune checkpoints are regulators of the immune system that are crucial for self-tolerance to prevent the immune system from attacking cells indiscriminately. Immune checkpoint can be inhibitory checkpoint molecules (e.g., favoring or inducing immune tolerance) or stimulatory checkpoint molecules (e.g., favoring or inducing immune response).

Throughout tumor progression, the immune system exerts a strong selective pressure, leading to immune tumor editing. As a result, malignant tumors often co-opt immune suppressive and tolerance mechanisms to avoid immune destruction. Immune checkpoint blockade inhibits T cell-negative co-stimulation in order to unleash antitumor T-cell responses that recognize tumor antigens. Inhibitory checkpoint molecules are therefore targeted for cancer immunotherapy due to their potential for use in multiple types of cancers.

Immune checkpoints of inhibitory pathways are fundamental in the immune system to maintain self-tolerance and modulate immune responses. Different immune cells are present in the tumor microenvironment. The expression immune cells ligand (by cancer cells) and the immune cells ligand-receptor interactions and secreted stimulatory growth factors, chemokines and cytokines are important in circumventing immune recognition or to immobilize effector T cells. The expression of these ligands and receptors by cancer cells provides some cancers with protect from attack by stimulating immune checkpoint targets.

Inhibitory checkpoint molecules include adenosine A2A receptor (A2AR); B7-H3 and B7-H4; B and T Lymphocyte Attenuator (BTLA); cytotoxic T-lymphocyte-associated protein 4 (CTLA-4); indoleamine 2,3-dioxygenase (IDO); killer-cell Immunoglobulin-like Receptor (KIR); lymphocyte activation gene-3 (LAG3); nicotinamide adenine dinucleotide phosphate NADPH oxidase isoform 2 (NOX2); programmed cell death 1 protein (PD-1) and its ligands PD-1 ligand 1 (PD-L1) and PD-L2; sialic acid-binding immunoglobulin-type lectin 7 (SIGLEC7); SIGLEC9; T-cell Immunoglobulin domain and Mucin domain 3 (TIM-3) and V-domain Ig suppressor of T cell activation (VISTA).

The Adenosine A2A receptor (A2AR) is an important checkpoint in cancer therapy because adenosine in the immune microenvironment, leads to the activation of the A2a receptor, induces a negative immune feedback loop and the tumor microenvironment has relatively high concentrations of adenosine.

B7 Homolog 3 (B7-H3) also known as cluster of differentiation 276 (CD276) is a human protein encoded by the CD276 gene. The B7-H3 protein is a 316 amino acid-long type I transmembrane protein existing in two isoforms determined by its extracellular domain. In mice, the extracellular domain consists of a single pair of immunoglobulin variable (IgV)-like and immunoglobulin constant (IgC)-like domains, whereas in humans it consists of one pair (2Ig-B7-H3) or two identical pairs (4lg-B7-H3) due to exon duplication. B7-H3 mRNA is expressed in most normal tissues. In contrast, B7-H3 protein has a very limited expression on normal tissues because of its post-transcriptional regulation by microRNAs. However, B7-H3 protein is expressed at high frequency on many different cancer types (60% of all cancers). In non-malignant tissues, B7-H3 has a predominantly inhibitory role in adaptive immunity, suppressing T cell activation and proliferation. In malignant tissues, B7-H3 is an immune checkpoint molecule that inhibits tumor antigen-specific immune responses. B7-H3 also possesses non-immunological pro-tumorigenic functions such as promoting migration, invasion, angiogenesis, chemoresistance, epithelial-to-mesenchymal transition, and affecting tumor cell metabolism. Due to its selective expression on solid tumors and its pro-tumorigenic function, B7H3 is the target of several anti-cancer agents including enoblituzumab, omburtamab, MGD009, MGC018, DS-7300a, and CAR T cells.

B7-H4, also called VTCNI (V-set domain-containing T-cell activation inhibitor 1) belongs to the B7 family of co-stimulatory proteins. B7-H4 is expressed by tumor cells and tumor-associated macrophages and plays a role in tumor escape by interacting with ligands expressed by T-lymphocytes.

B and T Lymphocyte Attenuator (BTLA) also known as CD272 is a surface protein whose expression is induced during the activation of T cells and remains on Th1 cells but not Th2 cells. Surface expression of BTLA is gradually downregulated during differentiation of human CD8+T cells from the naive to effector cell phenotype, however tumor specific human CD8+T cells express high levels of BTLA. Like programmed cell death 1 (PD1) and cytotoxic T-lymphocyte associate protein 4 (CTLA4), BTLA activates inhibitory pathways, regulating T cell activation. However, unlike PD-1 and CTLA-4, BTLA displays T-cell inhibition via interaction with tumor necrosis family receptors (TNF-R), not the B7 family of cell surface receptors. BTLA is a ligand for tumor necrosis factor (receptor) superfamily, member 14 (TNFRSF14), also known as herpes virus entry mediator (HVEM). BTLA-HVEM complexes negatively regulate T-cell immune responses.

CTLA4 or CTLA-4 (cytotoxic T-lymphocyte-associated protein 4), also known as CD152 (cluster of differentiation 152), is a protein receptor that, functioning as an immune checkpoint, downregulates immune responses. CTLA4 is constitutively expressed in regulatory T cells but only upregulated in conventional T cells after activation—a phenomenon which is particularly notable in cancers. CTLA4 is a member of the immunoglobulin superfamily that is expressed by activated T cells and transmits an inhibitory signal to T cells. CTLA4 is homologous to the T-cell co-stimulatory protein, CD28, and both molecules bind to CD80 and CD86, also called B7-1 and B7-2 respectively, on antigen-presenting cells. CTLA-4 binds CD80 and CD86 with greater affinity and avidity than CD28 thus enabling it to outcompete CD28 for its ligands. CTLA4 transmits an inhibitory signal to T cells, whereas CD28 transmits a stimulatory signal. CTLA4 is also found in regulatory T cells and contributes to its inhibitory function. T cell activation through the T cell receptor and CD28 leads to increased expression of CTLA-4.

Indoleamine 2,3-dioxygenase (IDO) is a tryptophan catabolic enzyme with immune-inhibitory properties. IDO is an immunomodulatory enzyme produced by alternatively activated macrophages and other immunoregulatory cells. IDO suppresses T and NK cells, generate Tregs and myeloid-derived suppressor cells, and also supports angiogenesis, and is therefore an immune checkpoint molecule. IDO allows tumor cells to escape the immune system by two main mechanisms. The first mechanism is based on tryptophan depletion from the tumor microenvironment, which leads to immune suppression. The second mechanism is based on the production of catabolic products called kynurenins, that are cytotoxic for T lymphocytes and NK cells. Overexpression of human IDO (hIDO) is described in a variety of human tumor cell lineages and is often associated with poor prognosis. Tumors with increased production of IDO include prostate, ovarian, lung or pancreatic cancer or acute myeloid leukemia.

Killer-cell immunoglobulin-like receptors (KIRs), are a family of type I transmembrane glycoproteins expressed on the plasma membrane of NK cells and a minority of T cells. KIRs regulate the killing function of these cells by interacting with major histocompatibility (MHC) class I molecules, which are expressed on all nucleated cell types. KIR receptors can distinguish between major histocompatibility (MHC) class I allelic variants, which allows them to detect virally infected cells or transformed cells. Most KIRs are inhibitory, meaning that their recognition of MHC molecules suppresses the cytotoxic activity of their NK cell.

Lymphocyte activation gene-3 (LAG3), also known as CD223 is a cell surface molecule with diverse biologic effects on T cell function. LAG3's main ligand is MHC class II, to which it binds with higher affinity than CD4. The protein negatively regulates cellular proliferation, activation, and homeostasis of T cells, in a similar fashion to CTLA-4 and PD-1 and has been reported to play a role in Treg suppressive function. LAG3 also helps maintain CD8+ T cells in a tolerogenic state and, working with PD-1, helps maintain CD8 exhaustion during chronic viral infection. LAG3 is known to be involved in the maturation and activation of dendritic cells.

Nicotinamide adenine dinucleotide phosphate NADPH oxidase isoform 2, also known as cytochrome b(558) subunit beta or Cytochrome b-245 heavy chain is an enzyme of myeloid cells that generates immunosuppressive reactive oxygen species. Genetic and pharmacological inhibition of NOX2 in myeloid cells improves anti-tumor functions of adjacent NK cells and T cells and also triggers autoimmunity in humans and experimental animals.

Programmed death I receptor or (PD-1) is an immune checkpoint that guards against autoimmunity through two mechanisms. First, it promotes apoptosis (programmed cell death) of antigen-specific T-cells in lymph nodes. Second, it reduces apoptosis in regulatory T cells (anti-inflammatory, suppressive T cells). PD-1 signalization relies on its interaction with one of its two ligands, PD-L1 and PD-L2. An advantage of targeting PD-1 is that it can restore immune function in the tumor microenvironment.

T-cell immunoglobulin domain and mucin domain 3 (TIM-3), also known as hepatitis A virus cellular receptor 2 (HAVCR2) is a cell surface molecule expressed on IFNγ producing CD4+ Th1 and CD8+ Tc1 cells, Th17 cells, regulatory T-cells, and innate immune cells (dendritic cells, NK cells, monocytes). TIM-3 acts as a negative regulator of Th1/Tc1 function by triggering cell death upon interaction with its ligand, galectin-9.

TIM-3is an immune checkpoint and together with other inhibitory receptors including PD-1 and LAG3 mediate the CD8+ T-cell exhaustion. TIM-3 has also been shown as a CD4+ Th1-specific cell surface protein that regulates macrophage activation and enhances the severity of experimental autoimmune encephalomyelitis in mice. TIM-3 expression is up regulated in tumor-infiltrating lymphocytes in lung, gastric, head and neck cancer, schwannoma, melanoma and follicular B-cell non-Hodgkin lymphoma.

V-domain Ig suppressor of T cell activation (VISTA) is a type I transmembrane protein that functions as an immune checkpoint. VISTA is produced at high levels in tumor-infiltrating lymphocytes, such as myeloid-derived suppressor cells and regulatory T cells, and its blockade with an antibody results in delayed tumor growth in mouse models of melanoma and squamous cell carcinoma. VISTA is primarily expressed on hematopoietic cells so that consistent expression of VISTA on leukocytes within tumors may allow VISTA blockade to be effective across a broad range of solid tumors.

Sialic acid-binding immunoglobulin-type lectin 7 (SIGLEC7), also known as CD328 and SIGLEC9 (also known as CD329) are proteins found on the surface of various immune cells, including natural killer cells and macrophages (SIGLEC7) and neutrophils, macrophages, dendritic cells and activated T-cells (SIGLEC9). SIGLECs 7 and 9 suppress the immune function of these cells by binding to terminal sialic acid on glycans that cover the surface of cells.

An “immune checkpoint inhibitor” or “checkpoint inhibitor therapy” is a form of cancer treatment that uses immune checkpoints which affect immune system functioning. Immune checkpoints can be stimulatory or inhibitory. Tumors can use these checkpoints to protect themselves from immune system attacks. Checkpoint therapy can block inhibitory checkpoints, restoring immune system function.

In various aspects, the immune checkpoint inhibitor is selected from the group consisting of PD-1 inhibitor; PD-L1 inhibitor; PD-L2 inhibitor; CTLA-4 inhibitor; A2AR inhibitor; B7-H3 inhibitor; B7-H4 inhibitor; BTLA; IDO inhibitor; KIR inhibitor; LAG3 inhibitor; NOX2 inhibitor; SIGLEC7 inhibitor; SIGLEC9 inhibitor; TIM-3 inhibitor; and VISTA inhibitor.

There are several checkpoint inhibitors that are currently used to treat cancer. PD-1 inhibitors include Pembrolizumab (Keytruda) and Nivolumab (Opdivo). PD-L1 inhibitors include Atezolizumab (Tecentriq), Avelumab (Bavencio) and Durvalumab (Imfinzi). CTLA-4 inhibitors include Iplimumab (Yervoy). There are several other checkpoint inhibitors being developed including an anti B7-H3 antibody (MGA271), an anti-KIR antibody (Lirilumab) and an anti-LAG3 antibody (BMS-986016).

Sequences:

SEQ ID NO: 1 PD-L1 TriKE amino acid sequence
QVQLVESGGGLVQPGGSLRLSCAASGLTFSSYNMGWFRQAPGQGLEAVASITWSGR
DTFYADSVKGRFTISRDNSKNTLYLQMNSLRAEDTAVYYCAANPWPVAAPRSGTYW
GQGTLVTVSSSGGGGSGGGGSGGGGSGGGGSGNWVNVISDLKKIEDLIQSMHIDATL
YTESDVHPSCKVTAMKCFLLELQVISLESGDASIHDTVENLIILANNSLSSNGNVTESG
CKECEELEEKNIKEFLQSFVHIVQMFINTSGSTSGSGKPGSGEGSTKGEIVLTQSPATLS
LSPGERATLSCRASQSVSSYLAWYQQKPGQAPRLLIYDASNRATGIPARFSGSGSGTD
FTLTISSLEPEDFAVYYCQQRSNWPTFGQGTKVEIKQVQLVQSGAEVKKPGSSVKVSC
KTSGDTESTYAISWVRQAPGQGLEWMGGIIPIFGKAHYAQKFQGRVTITADESTSTAY
MELSSLRSEDTAVYFCARKFHFVSGSPFGMDVWGQGTTVTVSS
SEQ ID NO: 2 CAM CD16
QVQLVESGGGLVQPGGSLRLSCAASGLTFSSYNMGWFRQAPGQGLEAVASITWSGR
DTFYADSVKGRFTISRDNSKNTLYLQMNSLRAEDTAVYYCAANPWPVAAPRSGTYW
GQGTLVTVSS
SEQ ID NO: 3 Linkerseq 16 Linker
SGGGGSGGGGSGGGGSGGGGSG
SEQ ID NO: 4 IL-15 (wild-type)
NWVNVISDLKKIEDLIQSMHIDATLYTESDVHPSCKVTAMKCFLLELQVISLESGDAS
IHDTVENLIILANNSLSSNGNVTESGCKECEELEEKNIKEFLQSFVHIVQMFINTS
SEQ ID NO: 5 Whitlow Linker
GSTSGSGKPGSGEGSTKG
SEQ ID NO: 6 anti-PD-L1 light chain (human)
EIVLTQSPATLSLSPGERATLSCRASQSVSSYLAWYQQKPGQAPRLLIYDASNRATGI
PARFSGSGSGTDFTLTISSLEPEDFAVYYCQQRSNWPTFGQGTKEVK
SEQ ID NO: 7 anti-PD-L1 heavy chain (human)
QVQLVQSGAEVKKPGSSVKVSCKTSGDTFSTYAISWVRQAPGQGLEWMGGIIPIFGK
AHYAQKFQGRVTITADESTSTAYMELSSLRSEDTAVYFCARKFHFVSGSPFGMDVW
GQGTTVTVSS
SEQ ID NO: 8 DNA encoding PD-L1 TriKE
caggtgcagctggtggagtctgggggaggcttggtgcagcctgggggctctctgagactctcctgtgcagcctctggcctcaccttcag
tagctataacatgggctggttccgccaggctccagggcaaggccttgaggctgtagcatctattacctggagtggtcgggacacattcta
tgcagactccgtgaagggccgattcaccatctccagagacaactccaagaacactctctatctgcaaatgaacagcctgcgcgcggag
gacacggccgtttattattgtgctgcaaacccctggccagtggcggcgccacgtagtggcacctactggggccaagggaccctggtca
ccgtctcctcatctggcggcggcggttctggtggaggaggtagtggggggggaggaagcggagggggtggctcagggaactgggt
gaatgtaataagtgatttgaaaaaaattgaagatcttattcaatctatgcatattgatgctactttatatacggaaagtgatgttcaccccagtt
gcaaagtaacagcaatgaagtgctttctcttggagttacaagttatttcacttgagtccggagatgcaagtattcatgatacagtagaaaatc
tgatcatcctagcaaacaacagtttgtcttctaatgggaatgtaacagaatctggatgcaaagaatgtgaggaactggaggaaaaaaatat
taaagaatttttgcagagttttgtacatattgtccaaatgttcatcaacacttctggcagtaccagcgggtcagggaaacctggcagtgggg
aaggttccacaaaaggtgaaattgtgttgacacagtctccagccaccctgtctttgtctccaggggaaagagccaccctctcctgcaggg
ccagtcagagtgttagcagctacttagcctggtaccaacagaaacctggccaggctcccaggctcctcatctatgatgcatccaacagg
gccactggcatcccagccaggttcagtggcagtgggtctgggacagacttcactctcaccatcagcagcctagagcctgaagattttgc
agtttattactgtcagcagcgtagcaactggccgacgttcggccaagggaccaaggtggaaatcaaacaggtccaactggtgcagtctg
gggctgaggtcaagaagcctgggtcgtcggtgaaggtctcctgcaagacttctggagacaccttcagcacctatgctatcagctgggtg
cgacaggcccctggacaagggcttgagtggatgggagggatcatccctatatttggtaaagcacactacgcacagaagttccagggca
gagtcacgattaccgcggacgaatccacgagcacagcctacatggagctgagcagcctgagatctgaggacacggccgtgtatttttgt
gcgagaaagtttcactttgtttcggggagccccttcggtatggacgtctggggccaagggaccacggtcaccgtctcctca
SEQ ID NO: 9DNA encoding CAM CD16
caggtgcagctggtggagtctgggggaggcttggtgcagcctgggggctctctgagactctcctgtgcagcctctggcctcaccttca
gtagctataacatgggctggttccgccaggctccagggcaaggccttgaggctgtagcatctattacctggagtggtcgggacacattc
tatgcagactccgtgaagggccgattcaccatctccagagacaactccaagaacactctctatctgcaaatgaacagcctgcgcgcgg
aggacacggccgtttattattgtgctgcaaacccctggccagtggcggcgccacgtagtggcacctactggggccaagggaccctgg
tcaccgtctcctca
SEQ ID NO: 10 DNA encoding Linker16 linker
tctggcggcggcggttctggtggaggaggtagtggggggggaggaagcggagggggtggctcaggg
SEQ ID NO: 11 DNA encoding IL-15 (wt)
aactgggtgaatgtaataagtgatttgaaaaaaattgaagatcttattcaatctatgcatattgatgctactttatatacggaaagtgatgttc
accccagttgcaaagtaacagcaatgaagtgctttctcttggagttacaagttatttcacttgagtccggagatgcaagtattcatgataca
gtagaaaatctgatcatcctagcaaacaacagtttgtcttctaatgggaatgtaacagaatctggatgcaaagaatgtgaggaactggag
gaaaaaaatattaaagaatttttgcagagttttgtacatattgtccaaatgttcatcaacacttct
SEQ ID NO: 12 DNA encoding Whitlow Linker
ggcagtaccagcgggtcagggaaacctggcagtggggaaggttccacaaaaggt
SEQ ID NO: 13 DNA encoding anti-PD-L1 Light chain
gaaattgtgttgacacagtctccagccaccctgtctttgtctccaggggaaagagccaccctctcctgcagggccagtcagagtgttag
cagctacttagcctggtaccaacagaaacctggccaggctcccaggctcctcatctatgatgcatccaacagggccactggcatccca
gccaggttcagtggcagtgggtctgggacagacttcactctcaccatcagcagcctagagcctgaagattttgcagtttattactgtcag
cagcgtagcaactggccgacgttcggccaagggaccaaggtggaaatcaaa
SEQ ID NO: 14 DNA encoding anti-PD-L1 Heavy chain
caggtccaactggtgcagtctggggctgaggtcaagaagcctgggtcgtcggtgaaggtctcctgcaagacttctggagacaccttca
gcacctatgctatcagctgggtgcgacaggcccctggacaagggcttgagtggatgggagggatcatccctatatttggtaaagcaca
ctacgcacagaagttccagggcagagtcacgattaccgcggacgaatccacgagcacagcctacatggagctgagcagcctgagat
ctgaggacacggccgtgtatttttgtgcgagaaagtttcactttgtttcggggagccccttcggtatggacgtctggggccaagggacc
acggtcaccgtctcctca
SEQ ID NO: 15 PD-L1 TriKE amino acid sequence
QVQLVESGGGLVQPGGSLRLSCAASGLTFSSYNMGWFRQAPGQGLEAVASITWSGR
DTFYADSVKGRFTISRDNSKNTLYLQMNSLRAEDTAVYYCAANPWPVAAPRSGTYW
GQGTLVTVSSPSGQAGAAASESLFVSNHAYNWVNVISDLKKIEDLIQSMHIDATLYTE
SDVHPSCKVTAMKCFLLELQVISLESGDASIHDTVENLIILANNSLSSNGNVTESGCKE
CEELEEKNIKEFLQSFVHIVQMFINTSEASGGPEEIVLTQSPATLSLSPGERATLSCRAS
QSVSSYLAWYQQKPGQAPRLLIYDASNRATGIPARFSGSGSGTDFTLTISSLEPEDFAV
YYCQQRSNWPTFGQGTKVEIKQVQLVQSGAEVKKPGSSVKVSCKTSGDTESTYAISW
VRQAPGQGLEWMGGIIPIFGKAHYAQKFQGRVTITADESTSTAYMELSSLRSEDTAV
YFCARKFHFVSGSPFGMDVWGQGTTVTVSS
SEQ ID NO: 16 HMA Linker
PSGQAGAAASESLFVSNHAY
SEQ ID NO: 17 Linker
EASGGPE
SEQ ID NO: 18 DNA encoding PD-L1 TriKE
caggtgcagctggtggagtctgggggaggcttggtgcagcctgggggctctctgagactctcctgtgcagcctctggcctcaccttcag
tagctataacatgggctggttccgccaggctccagggcaaggccttgaggctgtagcatctattacctggagtggtcgggacacattcta
tgcagactccgtgaagggccgattcaccatctccagagacaactccaagaacactctctatctgcaaatgaacagcctgcgcgcggag
gacacggccgtttattattgtgctgcaaacccctggccagtggcggcgccacgtagtggcacctactggggccaagggaccctggtca
ccgtctcctcaccgtctggtcaggctggtgctgctgctagcgaatctctgttcgtttctaaccacgcttacaactgggtgaatgtaataagtg
atttgaaaaaaattgaagatcttattcaatctatgcatattgatgctactttatatacggaaagtgatgttcaccccagttgcaaagtaacagc
aatgaagtgctttctcttggagttacaagttatttcacttgagtccggagatgcaagtattcatgatacagtagaaaatctgatcatcctagca
aacaacagtttgtcttctaatgggaatgtaacagaatctggatgcaaagaatgtgaggaactggaggaaaaaaatctgatcatcctagca
agagttttgtacatattgtccaaatgttcatcaacacttctgaagcttccggaggtcccgaggaaattgtgttgacacagtctccagccacc
ctgtctttgtctccaggggaaagagccaccctctcctgcagggccagtcagagtgttagcagctacttagcctggtaccaacagaaacct
ggccaggctcccaggctcctcatctatgatgcatccaacagggccactggcatcccagccaggttcagtggcagtgggtctgggacag
acttcactctcaccatcagcagcctagagcctgaagattttgcagtttattactgtcagcagcgtagcaactggccgacgttcggccaagg
gaccaaggtggaaatcaaacaggtccaactggtgcagtctggggctgaggtcaagaagcctgggtcgtcggtgaaggtctcctgcaa
gacttctggagacaccttcagcacctatgctatcagctgggtgcgacaggcccctggacaagggcttgagtggatgggagggatcatc
cctatatttggtaaagcacactacgcacagaagttccagggcagagtcacgattaccgcggacgaatccacgagcacagcctacatgg
agctgagcagcctgagatctgaggacacggccgtgtatttttgtgcgagaaagtttcactttgtttcggggagccccttcggtatggacgt
ctggggccaagggaccacggtcaccgtctcctca
SEQ ID NO: 19 DNA encoding HMA linker
ccgtctggtcaggctggtgctgctgctagcgaatctctgttcgtttctaaccacgcttac
SEQ ID NO: 20 DNA encoding Linker
gaagcttccggaggtcccgag
SEQ ID NO: 21 Altor IL-15 N72D mutation amino acid sequence
NWVNVISDLKKIEDLIQSMHIDATLYTESDVHPSCKVTAMKCFLLELQVISLESGDAS
IHDTVENLIILANDSLSSNGNVTESGCKECEELEEKNIKEFLQSFVHIVQMFINTS
SEQ ID NO: 22 Altor IL-15 N72A mutation amino acid sequence
NWVNVISDLKKIEDLIQSMHIDATLYTESDVHPSCKVTAMKCFLLELQVISLESGDAS
IHDTVENLIILANASLSSNGNVTESGCKECEELEEKNIKEFLQSFVHIVQMFINTS
SEQ ID NO: 23 human CD16 amino acid sequence
MEVQLVESGGGVVRPGGSLRLSCAASGFTFDDYGMSWVRQAPGKGLEWVSGINWN
GGSTGYADSVKGRFTISRDNAKNSLYLQMNSLRAEDTAVYYCARGRSLLFDYWGQ
GTLVTVSRGGGGSGGGGSGGGGSSELTQDPAVSVALGQTVRITCQGDSLRSYYASW
YQQKPGQAPVLVIYGKNNRPSGIPDRFSGSSSGNTASLTITGAQAEDEADYYCNSRDS
SGNHVVFGGGTKLTVL
SEQ ID NO: 24 DNA encoding human CD16
atggaagtgcagctggtggaaagcggcggcggcgtggtgcgcccgggcggcagcctgcgcctgagctgcgcggcgagcggcttt
acctttgatgattatggcatgagctgggtgcgccaggcgccgggcaaaggcctggaatgggtgagcggcattaactggaacggcgg
cagcaccggctatgcggatagcgtgaaaggccgctttaccattagccgcgataacgcgaaaaacagcctgtatctgcagatgaacag
cctgcgcgcggaagataccgcggtgtattattgcgcgcgcggccgcagcctgctgtttgattattggggccagggcaccctggtgac
cgtgagccgcggcggcggcggcagcggcggcggcggcagcggcggcggcggcagcagcgaactgacccaggatccggcggt
gagcgtggcgctgggccagaccgtgcgcattacctgccagggcgatagcctgcgcagctattatgcgagctggtatcagcagaaac
cgggccaggcgccggtgctggtgatttatggcaaaaacaaccgcccgagcggcattccggatcgctttagcggcagcagcagcgg
caacaccgcgagcctgaccattaccggcgcgcaggcggaagatgaagcggattattattgcaacagccgcgatagcagcggcaac
catgtggtgtttggcggcggcaccaaactgaccgtgctg
SEQ ID NO: 25 HLE sequence 1
DKTHTCPPCPAPELLGGPSVFLFPPKPKDTLMISRTPEVTCVWDVSHEDPEVKENWY
VDGVEVHNAKTKPCEEQYNSYRCVSVLTVLHQDWLNGKEYKCKVSNKALPAPIEK
TISKAKGQPREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKSLSLSPGG
GGSGGGGSGGGGSGGGGSGGGGSGGGGSDKTHTCPPCPAPELLGGPSVFLFPPKPKD
TLMISRTPEVTCWVDVSHEDPEVKFNWYDGVEVHNAKTKPCEEQYNSTYRCVSVLT
VLHQDWLNGKEYKCKVSNKALPAPIEKTISKAKGQPREPQVYTLPPSREEMKNQVS
LTCLVKGFYPSDIAVEWESNGQPENNYKTTPPVLDSDGSFFLYSKLTVDKSRWQQG
NVFSCSVMHEALHNHYTQKSLSLSP
SEQ ID NO: 26 HLE sequence 2
DKTHTCPPCPAPELLGGPSVFLFPPKPKDTLMISRTPEVTCVWDVSHEDPEVKENWY
VDGVEVHNAKTKPCEEQYNSTYRCVSVLTVLHQDWLNGKEYKCKVSNKALPAPIE
KTISKAKGQPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQKSLSLSPG
KGGGGSGGGGSGGGGSGGGGSGGGGSGGGGSDKTHTCPPCPAPELLGGPSVFLFPP
KPKDTLMISRTPEVTCWVDVSHEDPEVKFNWYVDGVEVHNAKTKPCEEQYNSTYR
CVSVLTVLHQDWLNGKEYKCKVSNKALPAPIEKTISKAKGQPREPQVYTLPPSREEM
TKNQVSLTCLVKGFYPSDIAVEWESNGQPENNYKTTPPVLDSDGSFFLYSKLTVDKS
RWQQGNVFSCSVMHEALHNHYTQKSLSLSPGK
SEQ ID NO: 27 HLE sequence 3
DKTHTCPPCPAPELLGGPSVFLFPPKPKDTLMISRTPEVTCWVDVSHEDPEVKFNWY
VDGVEVHNAKTKPCEEQYGSTYRCVSVLTVLHQDWLNGKEYKCKVSNKALPAPIE
KTISKAKGQPREPQVYTLPPSRKEMTKNQVSLTCLVKGFYPSDIAVEWESNGQPENN
YKTTPPVLKSDGSFFLYSKLTVDKSRWQQGNVFSCSVMHEALHNHYTQKSLSLSPGK
SEQ ID NO: 28 LE sequence 4
DKTHTCPPCPAPELLGGPSVFLFPPKPKDTLMISRTPEVTCVWDVSHEDPEVKENWY
VDGVEVHNAKTKPCEEQYGSTYRCVSVLTVLHQDWLNGKEYKCKVSNKALPAPIE
KTISKAKGQPREPQVYTLPPSREEMTKNQVSLTCLVKGFYPSDIAVEWESNGQPENN
YDTTPPVLDSDGSFFLYSDLTVDKSRWQQGNVFSCSVMHEALHNHYTQKSLSLSPGK
SEQ ID NO: 29 HLE sequence 5
DKTHTCPPCPAPELLGGPSVFLFPPKPKDTLMISRTPEVTCWVDVSHEDPEVKENWY
VDGVEVHNAKTKPCEEQYGSTYRCVSVLTVLHQDWLNGKEYKCKVSNKALPAPIE
KTISKAKGQPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQKSLSLSPG
KGGGGSGGGGSGGGGSGGGGSGGGGSGGGGSDKTHTCPPCPAPELLGGPSVFLFPP
KPKDTLMISRTPEVTCVWDVSHEDPEVKFNWYVDGVEVHNAKTKPCEEQYGSTYR
CVSVLTVLHQDWLNGKEYKCKVSNKALPAPIEKTISKAKGQPREPQVYTLPPSREEM
TKNQVSLTCLVKGFYPSDIAVEWESNGQPENNYKTTPPVLDSDGSFFLYSKLTVDKS
RWQQGNVFSCSVMHEALHNHYTQKSLSLSPGK
SEQ ID NO: 30 Fc region 1
DKTHTCPPCPAPELLGGPSVFLFPPKPKDTLMISRTPEVTCVWDVSHEDPEVKFNWY
VDGVEVHNAKTKPCEEQYNSYRCVSVLTVLHQDWLNGKEYKCKVSNKALPAPIEK
TISKAKGQPREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKSLSLSP
SEQ ID NO: 31 Fc region 2
DKTHTCPPCPAPELLGGPSVFLFPPKPKDTLMISRTPEVTCWVDVSHEDPEVKFNWY
DGVEVHNAKTKPCEEQYNSTYRCVSVLTVLHQDWLNGKEYKCKVSNKALPAPIEK
TISKAKGQPREPQVYTLPPSREEMKNQVSLTCLVKGFYPSDIAVEWESNGQPENNYK
TTPPVLDSDGSFFLYSKLTVDKSRWQQGNVFSCSVMHEALHNHYTQKSLSLSP
SEQ ID NO: 32 Fc region 3
DKTHTCPPCPAPELLGGPSVFLFPPKPKDTLMISRTPEVTCVWDVSHEDPEVKENWY
VDGVEVHNAKTKPCEEQYNSTYRCVSVLTVLHQDWLNGKEYKCKVSNKALPAPIE
KTISKAKGQPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQKSLSLSPGK
SEQ ID NO: 33 Fc region 4
DKTHTCPPCPAPELLGGPSVFLFPPKPKDTLMISRTPEVTCWVDVSHEDPEVKFNWY
VDGVEVHNAKTKPCEEQYNSTYRCVSVLTVLHQDWLNGKEYKCKVSNKALPAPIE
KTISKAKGQPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQKSLSLSPGK
SEQ ID NO: 34 Fc region 5
DKTHTCPPCPAPELLGGPSVFLFPPKPKDTLMISRTPEVTCWVDVSHEDPEVKFNWY
VDGVEVHNAKTKPCEEQYGSTYRCVSVLTVLHQDWLNGKEYKCKVSNKALPAPIE
KTISKAKGQPREPQVYTLPPSRKEMTKNQVSLTCLVKGFYPSDIAVEWESNGQPENN
YKTTPPVLKSDGSFFLYSKLTVDKSRWQQGNVFSCSVMHEALHNHYTQKSLSLSPGK
SEQ ID NO: 35 Fc region 6
DKTHTCPPCPAPELLGGPSVFLFPPKPKDTLMISRTPEVTCVWDVSHEDPEVKFNWY
VDGVEVHNAKTKPCEEQYGSTYRCVSVLTVLHQDWLNGKEYKCKVSNKALPAPIE
KTISKAKGQPREPQVYTLPPSREEMTKNQVSLTCLVKGFYPSDIAVEWESNGQPENN
YDTTPPVLDSDGSFFLYSDLTVDKSRWQQGNVFSCSVMHEALHNHYTQKSLSLSPGK
SEQ ID NO: 36 Fc region 7
DKTHTCPPCPAPELLGGPSVFLFPPKPKDTLMISRTPEVTCWVDVSHEDPEVKENWY
VDGVEVHNAKTKPCEEQYGSTYRCVSVLTVLHQDWLNGKEYKCKVSNKALPAPIE
KTISKAKGQPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQKSLSLSPGK
SEQ ID NO: 37 Fc region 8
DKTHTCPPCPAPELLGGPSVFLFPPKPKDTLMISRTPEVTCVWDVSHEDPEVKFNWY
VDGVEVHNAKTKPCEEQYGSTYRCVSVLTVLHQDWLNGKEYKCKVSNKALPAPIE
KTISKAKGQPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQKSLSLSPGK
SEQ ID NO: 38 scFc linker 1
GGGGSGGGGSGGGGSGGGGSGGGGSGGGGS
SEQ ID NO: 39 scFc linker 2
SSGGGGSGGGGSGGGGS

Although the invention has been described with reference to the above examples, it will be understood that modifications and variations are encompassed within the spirit and scope of the invention. Accordingly, the invention is limited only by the following claims.

1. An isolated nucleic acid sequence as set forth in SEQ ID NO:13 or 14 or a sequence having 90% identity thereto.

2. A protein encoded by a nucleic acid sequence of claim 1.

3. The protein of claim 2, wherein the amino acid sequence is selected from SEQ ID NO:6 or 7.

4. A fusion protein comprising the amino acid sequence set forth in SEQ ID NO: 6 and 7, operably linked to each other in either orientation.

5. The fusion protein of claim 4, wherein the protein comprises SEQ ID NO: 6 and 7, in direct linkage between the C-terminus of SEQ ID NO:6 and the N-terminus of SEQ ID NO:7.

6. The fusion protein of claim 4, wherein the protein comprises SEQ ID NO: 7 and 6, in direct linkage between the C-terminus of SEQ ID NO:7 and the N-terminus of SEQ ID NO:6.

7. A fusion protein comprising the sequence set forth in SEQ ID NO:1 or 15 and sequences having 90% or greater identity to SEQ ID NO:1 or 15.

8. A fusion protein comprising in operably linkage, SEQ ID NO:2 or 23; 4, 21 or 22; 6 and 7 or 7 and 6.

9. The fusion protein of claim 8, wherein SEQ ID NO:2 or 23 and 4, 21 or 22 are linked by SEQ ID NO:3 or SEQ ID NO:16.

10. The fusion protein of claim 8, wherein SEQ ID NO:4, 17 or 18 and 6 or 7 are linked by SEQ ID NO:5 or SEQ ID NO:17.

11. The fusion protein of claim 8, wherein SEQ ID NO:6 and 7 are in operable linkage in either orientation.

12. The fusion protein of claim 8, further comprising a half-life extending (HLE) molecule.

13. The fusion protein of claim 12, wherein the HLE molecule is a Fc or a scFc antibody fragment comprising any one of SEQ ID NOs: 25-29.

14. The fusion protein of claim 8, wherein SEQ ID NO:4 has an N72 substitution.

15. The fusion protein of claim 14, wherein the N72 mutation is N72A or N72D.

16. The fusion protein of claim 15, wherein the protein is set forth in SEQ ID NO: 21 or 22.

17. An isolated nucleic acid sequence encoding the fusion protein of claim 4.

18. The isolated nucleic acid sequence of claim 17, wherein the sequence is SEQ ID NO:8 or SEQ ID NO: 18.

19. A method of treating cancer in a subject comprising administering to the subject a fusion protein of claim 4, thereby treating the cancer.

20. The method of claim 19, further comprising administering to the subject an immune checkpoint inhibitor.

21. The method of claim 20, wherein the immune checkpoint inhibitor is selected from the group consisting of programmed cell death 1 protein (PD-1) inhibitor, PD-1 ligand 1 (PD-L1) inhibitor, PDD-L2 inhibitor, cytotoxic T-lymphocyte-associated protein 4 (CTLA-4) inhibitor, Adenosine A2A receptor (A2AR) inhibitor, B7-H3 inhibitor, B7-H4 inhibitor, B and T Lymphocyte Attenuator (BTLA) inhibitor, Indoleamine 2,3-dioxygenase (IDO) inhibitor, Killer-cell Immunoglobulin-like Receptor (KIR) inhibitor, Lymphocyte Activation Gene-3 (LAG3) inhibitor, nicotinamide adenine dinucleotide phosphate NADPH oxidase isoform 2 (NOX2) inhibitor, sialic acid-binding immunoglobulin-type lectin 7 (SIGLEC7) inhibitor, SIGLEC9 inhibitor, T-cell Immunoglobulin domain and Mucin domain 3 (TIM-3) inhibitor, and V-domain Ig suppressor of T cell activation (VISTA) inhibitor.

22. The method of claim 19, wherein the cancer is selected from non-small lung cancer, cutaneous squamous cell carcinoma, pancreatic cancer, primary hepatocellular carcinoma, colorectal carcinoma, clear cell renal carcinoma, prostate cancer, cervical cancer, ovarian cancer, melanoma, brain cancer, leukemia, lymphoma, myeloma, head and neck cancer or breast cancer.

23. A fusion protein comprising SEQ ID NO:23, SEQ ID NO:21 or 22 and SEQ ID NO:6 and 7 in either orientation.

24. The fusion protein of claim 23, wherein SEQ ID NO:23 is operably linked to SEQ ID NO:21 or 22 by a linker of SEQ ID NO:3 or 16.

25. The fusion protein of claim 23, wherein SEQ ID NO:21 or 22 is operably linked to SEQ 6 and 7, in either orientation by a linker of SEQ ID NO:5 or 17.

26. The fusion protein of claim 23, further comprising a half-life extending (HLE) molecule.

27. The fusion protein of claim 26, wherein the HLE molecule is a Fc or a scFc antibody fragment comprising any one of SEQ ID NOs: 25-29.
